(19) United States
(12) Patent Application Publication
(10) Pub. No.: US 2016/0228452 A1
(43) Pub. Date: Aug. 11, 2016

(54) TREATMENT OF METABOLIC DISEASES BY INHIBTION OF HTR2A OR HTR3

(71) Applicant: Korea Advanced Institute of Science and Technology, Daejeon (KR)

(72) Inventors: Ha Il KIM, Daejeon (KR); Sang Kyu PARK, Gangneung (KR)

(21) Appl. No.: 14/611,143

(22) Filed: Jan. 30, 2015

(30) Foreign Application Priority Data
Mar. 28, 2014 (KR) 10-2014-0036708

(51) Int. Cl. A61K 31/554; A61K 31/519; A61K 31/496; A61K 31/551; A61K 31/407; A61K 31/445; A61K 31/4178; A61K 31/46; A61K 31/439; A61K 31/473; A61K 31/4184; A61K 31/437; A61K 31/166; A61K 45/06; G01N 33/50; C12Q 1/68; A61K 31/517

(52) U.S. Cl. CPC A61K 31/554; A61K 31/517; A61K 31/519; A61K 31/496; A61K 31/551; A61K 31/407; A61K 31/445; A61K 31/4178; A61K 31/46; A61K 31/439; A61K 31/473; A61K 31/4184; A61K 31/437; A61K 31/166; A61K 45/06; G01N 33/5044; C12Q 1/6883; C12Q 2600/158; C12Q 2600/136

(57) ABSTRACT

The present invention relates to a method for preventing or treating a metabolic disease, which comprises inhibiting HTR2A (5-hydroxytryptamine 2A receptor) or HTR3A (5-hydroxytryptamine 3A receptor). The inhibition of HTR3A leads to various advantageous efficacies for therapy of metabolic diseases, including resistance to obesity, decrease of lipid droplet size, increase of expression levels of thermogenic genes, increase of metabolic activities, increase of insulin sensitivity and decrease of LDL cholesterol and leptin.

### CROSS-REFERENCE TO RELATED APPLICATIONS

This application claims priority under 35 U.S.C. §119 to Korean Patent Application No. 10-2014-0036708, filed on Mar. 28, 2014, in the Korean Intellectual Property Office, the disclosure of which is incorporated herein by reference in its entirety.

### BACKGROUND OF THE INVENTION

1. Field of the Invention

The present invention relates to the treatment of metabolic diseases such as obesity by inhibition of 5-hydroxytryptamine 2a receptor (HTR2a) or 5-hydroxytryptamine 3 receptor (HTR3).

2. Description of the Related Art

Central 5-HT (5-hydroxytryptamine, serotonin) works as an anorexigenic neurotransmitter by activating the Htr2c receptor in POMC neurons¹⁻⁴. However, recent human genetic studies have reported the different relationship between 5-HT and obesity⁶⁻⁸. Mice with serotonin transporter (SERT) gene mutation were expected to be slim due to the increased 5-HT activity, but these mice showed an obese phenotype⁹. In contrast, the body weight was reduced in Tryptophan hydroxylase-1 (Tph1) and Tryptophan hydroxylase-2 (Tph2) KO mice¹⁰⁻¹². Leptin-deficient ob/ob mice show increased 5-HT levels in the gut compared with wild type (WT) littermates¹³. However, gut-derived 5-HT is not associated with diet-induced weight gain¹⁴.

Accordingly, there remain needs in the art to more exactly elucidate 5-HT as a target for anti-obesity treatments.

Throughout this application, various patents and publications are referenced, and citations are provided in parentheses. The disclosure of these patents and publications in their entities are hereby incorporated by references into this application in order to more fully describe this invention and the state of the art to which this invention pertains.

### SUMMARY OF THE INVENTION

The present inventors have made intensive researches to develop a novel strategy for prevention or treatment of metabolic diseases, particularly obesity. As a result, we have found that among various 5-HT (5-hydroxytryptamine) receptors, HTR2A (5-hydroxytryptamine 2a receptor) and HTR3 (5-hydroxytryptamine 3 receptor) are a promising therapeutic target for metabolic diseases, particularly obesity.

Accordingly, it is an object of this invention to provide a method for preventing or treating a metabolic disease.

It is another object of this invention to provide a method for screening a therapeutic agent for treating a metabolic disease.

Other objects and advantages of the present invention will become apparent from the detailed description to follow taken in conjugation with the appended claims and drawings.

### BRIEF DESCRIPTION OF THE DRAWINGS

FIGS. 1a-1h represents serotonin depletion protects mice from obesity. In FIG. 1a, serotonin levels in adipose tissues from C57BL/6J male mice after 16 weeks of high-fat diet (HFD) feeding, as assessed by LC-MS; n=4 mice per group. The HFD increased the serotonin levels in the epididymal white adipose tissue (eWAT) and inguinal white adipose tissue (iWAT). In FIG. 1b, Tph1•mRNA expression in adipose tissues was assessed via RT-PCR after 2 weeks of HFD administration. n=4 mice per group. FIG. 1s represents growth curves of control mice and PCPA-treated mice fed a standard chow diet (SCD) or HFD. n=4 mice per group. The PCPA-treated mice fed an HFD showed a reduction in body weight gain and decreased eWAT compared with the control mice fed an HFD. FIG. 1d represents gross images of SCD-fed or HFD-fed control mice and PCPA-treated mice after 10 weeks of HFD feeding. PCPA-treated mice fed an HFD showed decreased eWAT compared with control mice fed an HFD. In FIGS. 1e and 1f, PCPA-treated mice fed an HFD showed improved glucose tolerance (FIG. 1e) and reduced insulin resistance (FIG. 1f) compared with control mice fed a HFD. FIGS. 1g and 1h represents the metabolic rates of control and PCPA-treated mice fed an HFD for 6 weeks. The metabolic parameters were measured using an 8-chamber Oxymax system. Mice were acclimatized to cages for 24 hr, and data were collected for an additional 48 hr. The PCPA-treated mice showed increased carbon dioxide production (VCO₂) and heat production. n=4 mice per group. All data are presented as the mean±standard error. *P<0.05. LC-MS: Liquid chromatography-mass spectrophotometer.

FIG. 1i represents detection of Tph1 and 5-HT receptor mRNAs in mouse adipose tissues. Adipose tissues isolated from epididymal white adipose tissue (eWAT), inguinal white adipose tissue (iWAT) and interscapular brown adipose tissue (BAT) of C57BL/6J mice at 8 weeks of age. The Tph1 mRNA, 5-HT receptor (Htr) subtypes and other genes related to 5-HT metabolism were amplified from mouse adipose tissue RNA by RT-PCR.

FIG. 1j represents effects of 5-HT depletion on lipid profiles, leptin and adiponectin. Mice were treated with vehicle or PCPA (300 mg/kg) for 6 weeks from 8 weeks of age. During the treatment period, these mice were fed an SCD or HFD. After 6 weeks of treatment, blood was obtained from tail vein. Serum levels of total cholesterol (a), LDL cholesterol (b), FFA (c) and leptin (d) were measured by ELISA. PCPA-treated mice fed an HFD showed reduced cholesterol, leptin, and FFA levels compared with control mice fed an HFD. All data are presented as the mean±standard error. Statistical significance vs. untreated control was analyzed by Student's t test: *, P<0.05. Vehicle: polyethylene glycol 400. LDL: low density lipoprotein; FFA: free fatty acid; SCD: standard chow diet; HFD: high fat diet.

FIG. 1k represents effects of peripheral Tph1 inhibitor on body weight and glucose metabolism. Mice were treated orally with vehicle or LP-533401 (30 mg/kg) for 10 weeks from 8 weeks of age. During the treatment period, these mice were fed an SCD or HFD. (a) Growth curves of control mice and LP-533401-treated mice fed an SCD or a HFD. (b) IPGTT were performed after overnight fasting. Blood glucose levels were measured after intraperitoneal injection of 2 g/kg glucose. LP-533401-treated mice fed an HFD showed improved glucose tolerance compared with control mice fed an HFD. (c) IPITT was performed after fasted 4 hrs. Blood glucose levels were measured after intraperitoneal injection of 0.75 U/kg. n=4 mice per group. (d) Representative BAT sections from control and LP-533401-treated mice stained with hematoxylin and eosin (H&E). Scale bar indicates 20 μm. All data are presented as the mean±standard error. Statistical significance vs. untreated control was analyzed by Student's t test: *, P<0.05. LP-533401: small-molecule inhibitor of peripheral Tph; vehicle: polyethylene glycol 400; IPGTT: Intraperitoneal glucose tolerance test; IPITT: intraperitoneal insulin tolerance test.

FIG. 1l represents food intake and physical activity of PCPA-treated mice during HFD. The metabolic profiles of 14-week-old control and PCPA-treated mice were measured by Oxymax system, after fed a HFD for 6 weeks. Mice were acclimatized for 24 hrs, data were collected for 48 hrs. Daily food intake (a) and physical activity (b) were similar in both groups. (c) The O₂ consumption of PCPA-treated mice was increased. n=4 mice per group. All data are presented as the mean±standard error. Statistical significance vs. untreated control was analyzed by Student's t test: *, P<0.05. Vehicle: PBS; VO₂: O₂ consumption; RER: respiratory exchange ratio.

FIG. 1m represents food intake and metabolic rates of PCPA-treated mice during SCD. The metabolic profiles of 14-week-old SCD-fed mice were measured by Oxymax system, after treated with vehicle or PCPA (300 mg/kg) for 6 weeks. The metabolic cage data showed no significant differences between vehicle and PCPA-treated mice. The metabolic cage data included daily food intake (a), physical activity (b), heat production (c), oxygen consumption (d), VCO₂ elimination (e) and respiratory exchange ratio (f). n=4 mice per group. All data are presented as mean±standard error. Statistical significance vs. the untreated control was analyzed by Student's t test: *, p<0.05. Vehicle: PBS; VCO₂: CO₂ elimination.

FIGS. 2a-2i represent 5-HT depletion in adipose tissues in mice. (FIG. 2a) Representative images of eWAT of control mice and PCPA-treated mice after 8 weeks of SCD or HFD feeding. Sections were stained with hematoxylin and eosin (H&E). The scale bar indicates 20 μm. (FIG. 2b) Average adipocyte sizes measured from H&E-stained images. The HFD increased the adipocyte size in eWAT in control mice, but did not increase the adipocyte size in PCPA-treated mice. (FIG. 2c) Expression of genes associated with lipogenesis in eWAT, as assessed by RT-PCR. PCPA-treated mice showed suppressed expression of lipogenic genes in eWAT compared with control mice under both SCD feeding and HFD feeding conditions. (FIG. 2d, 2e) Representative images of iWAT stained with H&E (FIG. 2d) and with immunohistochemical staining for Ucp1 (FIG. 2e). Silver color means Ucp1-positive adipocytes. The scale bar indicates 20 μm. (FIG. 2f) Expression of genes associated with thermogenesis in iWAT, as assessed via RT-PCR. The Ucp1 and Dio2 levels were increased in PCPA-treated mice fed an HFD. (FIG. 2g) H&E staining of BAT from control mice and PCPA-treated mice. HFD administration increased the lipid droplet sizes in the BAT, but the PCPA-treated BAT showed decreased lipid droplet sizes compared with the control BAT, regardless of the diet consumed. The scale bar indicates 20 μm. (FIG. 2h) Expression of genes associated with thermogenesis in BAT, as assessed via RT-PCR. (FIG. 2i) Glucose utilization of BAT, as determined by ¹⁸F-FDG PET/CT. BAT from PCPA-treated mice fed an HFD showed increased glucose uptake compared with control mice fed an HFD. All data are presented as the mean±standard error. *p<0.05.

FIG. 2j shows representative section of the eWAT of PCPA-treated mice stained with the Plin1 antibody. Histology of eWAT from control and PCPA-treated mice after 6 weeks SCD or HFD feeding. Perlipin1 (Plin 1) immunohistochemical staining of eWAT revealed intact adipocytes in each group. No gross cell damage was observed in eWAT after PCPA treatment (300 mg/kg) for 6 weeks. The scale bar indicates 20 μm. Vehicle: PBS.

FIG. 2k represents effect of serotonin depletion on mouse eWAT. Mice were treated with vehicle or PCPA (300 mg/kg) for 6 weeks from 8 weeks of age. During the treatment period, these mice were fed an SCD or an HFD. After 6 weeks of treatment, eWAT were isolated and the mRNA levels of genes involved in lipid metabolism and thermogenesis were measured by real time RT-PCR. n=4 mice per group. All data are presented as the mean±standard error. Statistical significance vs. the untreated control was analyzed by Student's t test: *, P<0.05. Vehicle: polyethylene glycol 400.

FIG. 2l represents adipocyte size of iWAT. The average adipocyte size of H&E stained images were calculated using the Image J software. iWAT from 18-week-old PCPA-treated mice fed an HFD for 10 weeks shows decreased adipocyte size compared with PCPA-treated mice fed an SCD. n=4 mice per group. All data are presented as mean±standard error. Statistical significance vs. the untreated control was analyzed by Student's t test: *, P<0.05.

FIGS. 2m-2o represent effect of serotonin depletion on mouse iWAT. Mice were treated with vehicle or PCPA (300 mg/kg) for 6 weeks from 8 weeks of age. During the treatment period, these mice were fed an SCD or an HFD. After 6 weeks of treatment, iWAT were isolated and the mRNA levels of genes involved in lipid metabolism and thermogenesis were measured by real time RT-PCR. n=4 mice per group. All data are presented as the mean±standard error. Statistical significance vs. untreated control was analyzed by Student's t test: *, P<0.05.

FIGS. 2p-2r represent effect of serotonin depletion on mouse BAT. Mice were treated with vehicle or PCPA (300 mg/kg) for 6 weeks from 8 weeks of age. During the treatment period, these mice were fed an SCD or an HFD. After 6 weeks of treatment, BAT were isolated and the mRNA levels of genes involved in lipid metabolism and thermogenesis were measured by real time RT-PCR. n=4 mice per group. All data are presented as the mean±standard error. Statistical significance vs. untreated control was analyzed by Student's t test: *, P<0.05.

FIG. 2s shows representative images of PET-CT. BAT (triangle) and heart tissue (arrow) are highlighted. ¹⁸F-FDG uptake increased in BAT. BAT of WT mice showed decreased ¹⁸F-FDG uptake after HFD feeding but BAT from PCPA-treated mice fed an HFD showed increased glucose uptake compared with control mice fed an HFD.

FIG. 2t shows representative TEM images of mitochondria from PCPA-treated BAT. Interscapular BAT was isolated from SCD-fed mice, HFD-fed mice, HFD-fed mice with PCPA treatment for 6 weeks. BAT from HFD-fed mice with PCPA treatment shows increased size and number of mitochondria. The scale bar indicates 1 μm. TEM: transmission electron microscopy.

FIGS. 3a-3p address that Htr3 regulates thermogenesis in BAT, and Htr2a regulates lipogenesis in WAT. (FIG. 3a) Growth curves of Htr3a KO mice and their WT littermates fed an SCD or HFD. The Htr3a KO mice were resistant to obesity. n=4 mice per group. (FIG. 3b) H&E staining of BAT from WT and Htr3a KO mice after 6 weeks of SCD or HFD feeding. The BAT from the KO mice showed decreased lipid droplet sizes compared with those of the WT littermates. The scale bar indicates 20 μm. (FIG. 3c) Thermogenic gene expression levels in BAT. n=4 per group. (FIG. 3d, 3e) The metabolic rates of WT and Htr3a KO mice fed a HFD. The Htr3a KO mice showed increased carbon dioxide production (VCO₂) (FIG. 3d) and heat production (FIG. 3e). n=4 mice per group. (FIG. 3f, 3g) Glucose tolerance and insulin tolerance tests after 6 weeks of HFD administration. n=4 per group. (FIG. 3h) Changes in cAMP levels, as assessed using a cAMP ELISA following Htr3 agonist/antagonist treatment of immortalized brown adipocytes (IBAs). In the absence of the β3 adrenergic receptor (β3AR) agonist, the Htr3 agonist/antagonist did not affect cAMP levels in the brown adipocytes. However, after β3AR stimulation, Htr3 antagonist-pretreated IBAs showed increased cAMP levels. Ondan: ondansetron; CL: CL 316243. (FIG. 3i) Western blot assay for the phosphorylation of PKA pathway component. Htr3 antagonist-pretreated IBAs showed increased phosphorylation of PKA pathway components after β3AR stimulation. (FIG. 3j) Thermogenic gene expression levels were increased 2 h after ondansetron treatment. CL: CL 316243. (FIG. 3k) The Ucp1 mRNA level was decreased 2 h after m-CBPG (Htr3 agonist) treatment of IBAs. CL: CL-316243; m-CBPG: 1-(m-chlorophenyl)-biguanide. (FIG. 3l) Metabolic assays of IBAs using the Seahorse XF24 analyzer. After full differentiation, Htr3 antagonist pre-treatment increased the oxygen consumption rate of the IBAs following β3AR agonist stimulation. n=5 per group. (FIG. 3m) Expression of Htr2a in 3T3-L1 adipocytes. Htr2a mRNA was increased in fully differentiated 3T3-L1 adipocytes. The expression level was normalized to that measured at Day 0. (FIG. 3n) The relative mRNA expression after Htr2a agonist treatment of 3T3-L1 mature adipocytes. DOI: 2,5-dimethoxy-4-iodoamphetamine, Htr2a agonist. (FIG. 3o) Oil Red O staining of 3T3-L1 adipocytes. After Oil Red 0 staining of fully differentiated 3T3-L1 adipocytes, the stain was extracted from the cells, and the absorbance of the extract was measured spectrophotometrically. Treatment with the Htr2a agonist ketanserin during adipocyte differentiation reduced the optical density of the extract. (FIG. 3p) Glycerol release assay. 5-HT and DOI reduced the glycerol release of 3T3-L1 mature adipocytes. All data are presented as the mean±standard error. *p<0.05. Vehicle: PBS.

FIGS. 4a-4m represent cell autonomous function of 5-HT in adipocytes. (FIG. 4a) Body weight of Tph1 FKO mice and their WT littermates fed a HFD. n=4 per group. (FIG. 4b) Representative images of the adipose tissues of Tph1 FKO (adipose tissue specific Tph1 KO) mice and WT littermates after 6 weeks of HFD feeding. Sections were stained with H&E. (FIG. 4c) Immunohistochemistry for Ucp1 (blue color). The Ucp1-positive multilocular adipocytes were increased in the iWAT of Tph1 FKO mice. (FIG. 4d) Glucose tolerance test. HFD-fed Tph1 FKO mice showed improved glucose tolerance compared with HFD-fed WT littermates. (FIG. 4e) Ex vivo study using the SVF from the BAT of Tph1 FKO mice and WT littermates. After 8 days of differentiation, Tph1 FKO SV cells showed increased Ucp1 mRNA expression compared with WT SV cells. 5-HT suppressed the Ucp1 mRNA level in SV cells from Tph1 FKO mice. (FIG. 4f) Growth curves of Tph1 AFKO (Inducible adipocyte specific Tph1 KO in adult) mice and their WT littermates fed a HFD. n=4 per group. (FIG. 4g, 4h) Tph1 AFKO mice fed a HFD showed improved glucose tolerance (FIG. 4g) and reduced insulin resistance (FIG. 4h) compared with control mice after 6 weeks of HFD feeding. (FIG. 4i) Representative images of the adipose tissues of WT littermates and Tph1 AFKO mice after 6 weeks of HFD feeding. The sections were stained with H&E. (FIG. 4j) Average adipocyte sizes measured from the H&E stained images. (FIG. 4k) Immunohistochemistry for Ucp1 (blue color). (FIG. 4l, 4m) A proposed model for the regulation of energy metabolism by 5-HT in adipocytes. Red arrows denote activation and blue arrows denote suppression. All data are presented as the mean±standard error. *p<0.05.

### DETAILED DESCRIPTION

In an aspect of this invention, there is provide a method for preventing or treating a metabolic disease, which comprises inhibiting HTR2A (5-hydroxytryptamine 2a receptor) or HTR3 (5-hydroxytryptamine 3 receptor) in a subject in need thereof.

The present inventors have made intensive researches to develop a novel strategy for prevention or treatment of metabolic diseases, particularly obesity. As a result, we have found that among various 5-HT (5-hydroxytryptamine) receptors, HTR2A (5-hydroxytryptamine 2a receptor) and HTR3 (5-hydroxytryptamine 3 receptor) are a promising therapeutic target for metabolic diseases, particularly obesity.

According to the present invention, the prevention or treatment of metabolic diseases is accomplished by inhibiting HTR2A (5-hydroxytryptamine 2a receptor) or HTR3 (5-hydroxytryptamine 3 receptor).

HTR3 is well known as a heteropentamer of HTR3A and HTR3B that acts as a functional serotonin-gated cation channel^{23,24}.

The inhibition of HTR2A or HTR3 may be performed by various fashions or methods.

According to an embodiment, the inhibition of HTR2A or HTR3 is performed by suppressing the expression of HTR2A or HTR3 (particularly, HTR3A).

The expression of the HTR2A gene or the HTR3A gene may be suppressed by an antisense or siRNA oligonucleotide.

As used herein, the term “antisense oligonucleotide” refers to a DNA, an RNA or a derivative thereof including a nucleotide sequence complementary to a specific mRNA sequence, thus binding to the complementary sequence of the mRNA and inhibiting translation of the mRNA into a protein. The antisense sequence is a DNA or RNA sequence complementary to HTR2A mRNA (e.g., GenBank Accession Nos. NM_000621.4 and NM_001165947.2) or HTR3A mRNA (e.g., GenBank Accession Nos. NR_046363.1, NM_001161772.2, NM_000869.5 and NM_213621.3) and capable of binding to the HTR2A or HTR3A mRNA, thus inhibiting translation of the Htr2a or Htr3a mRNA, translocation into the cytoplasm, maturation, or any other activity essential to overall biological functions. The antisense nucleotide may be 6 to 100 bases in length, specifically 8 to 60 bases in length, more specifically 10 to 40 bases in length. The antisense oligonucleotide may be either synthesized in vitro and administered into the body or it may be synthesized in vivo. An example of synthesizing the antisense oligonucleotide in vitro is to use RNA polymerase I. An example of synthesizing the antisense oligonucleotide in vivo is to use a vector having the origin of the multiple cloning site (MCS) in opposite direction so that the antisense RNA is transcribed. Specifically, the antisense RNA may have a translation stop codon within its sequence in order to prevent translation into a peptide sequence.

As used herein, the term “siRNA” refers to a nucleotide molecule capable of mediating RNA interference (RNAi) or gene silencing (see WO 00/44895, WO 01/36646, WO 99/32619, WO 01/29058, WO 99/07409 and WO 00/44914). Since siRNA can suppress the expression of the target gene, it provides an effective way of gene knockdown or genetic therapy. First discovered in plants, worms, fruit flies and parasites, siRNA has been recently developed and used for studies of mammal cells.

In case the siRNA molecule is used in the present disclosure, it may have a structure in which its sense strand (a sequence corresponding to the HTR2A or HTR3A mRNA sequence) and its antisense strand (a sequence complementary to the HTR2A or HTR3A mRNA sequence) form a double strand. Alternatively, it may have a single-stranded structure having self-complementary sense and antisense strands.

The siRNA is not limited to those in which double-stranded RNA moieties constitute complete pairs, but includes the unpaired moieties such as mismatch (corresponding bases are not complementary), bulge (no base in one chain), etc. The total length of the siRNA may be 10 to 100 bases, specifically 15 to 80 bases, more specifically 20 to 70 bases. The end of the siRNA may be either blunt or cohesive as long as it is capable of suppressing the expression of the Htr2a or Htr3a gene via RNAi. The cohesive end may be either 3′- or 5′-end.

In the present disclosure, the siRNA molecule may have a short nucleotide sequence (e.g., about 5-15 nucleotides) inserted between the self-complementary sense and antisense strands. In this case, the siRNA molecule formed from the expression of the nucleotide sequence forms a hairpin structure via intramolecular hybridization, resulting in a stem-and-loop structure overall. The stem-and-loop structure is processed in vitro or in vivo to give an activated siRNA molecule capable of mediating RNAi.

According to an embodiment, the inhibition of HTR2A or HTR3 is performed by administering to the subject an antagonist against HTR2A or HTR3 (particularly, HTR3A).

A number of antagonists against HTR2A are well known to one of skill in the art. According to an embodiment, the antagonist against HTR2A useful in the present invention includes ketanserin [3-{2-[4-(4-fluorobenzoyl)piperidin-1-yl]ethyl}quinazoline-2,4(1H,3H)-dione], ritanserin [6-[2-[4-[bis(4-fluorophenyl)methylidene]piperidin-1-yl]ethyl]-7-methyl-[1,3]thiazolo[2,3-b]pyrimidin-5-one], nefazodone [1-(3-[4-(3-chlorophenyl)piperazin-1-yl]propyl)-3-ethyl-4-(2-phenoxyethyl)-1H-1,2,4-triazol-5(4H)-one], clozapine [8-Chloro-11-(4-methylpiperazin-1-yl)-5H-dibenzo[b,e][1,4]diazepine], olanzapine [2-Methyl-4-(4-methyl-1-piperazinyl)-10H-thieno[2,3-b][1,5]benzodiazepine], quetiapine [2-(2-(4-dibenzo[b,f][1,4]thiazepine-11-yl-1-piperazinyl)ethoxy)ethanol], risperidone [4-[2-[4-(6-fluorobenzo[d]isoxazol-3-yl)-1-piperidyl]ethyl]-3-methyl-2,6-diazabicyclo[4.4.0]deca-1,3-dien-5-one], asenapine [(3aRS,12bRS)-rel-5-Chloro-2,3,3a,12b-tetrahydro-2-methyl-1H-dibenz[2,3:6,7]oxepino[4,5-c]pyrrole], volinanserin [(R)-(2,3-dimethoxyphenyl)-[1-[2-(4-fluorophenyl)ethyl]-4-piperidyl]methanol], and AMDA [9-Aminomethyl-9,10-dihydroanthracene)].

Various antagonists against HTR3A are well known to one of skill in the art. According to an embodiment, the antagonist against HTR3A useful in the present invention includes ondansetron [(RS)-9-methyl-3-[(2-methyl-1H-imidazol-1-yl)methyl]-2,3-dihydro-1H-carbazol-4(9H)-one], granisetron [1-methyl-N-((1R,3r,5S)-9-methyl-9-azabicyclo[3.3.1]nonan-3-yl)-1H-indazole-3-carboxamide], tropisetron [(1R,5S)-8-methyl-8-azabicyclo[3.2.1]octan-3-yl 1methyl-indole-3-carboxylate], dolasetron [(3R)-10-oxo-8-azatricyclo[5.3.1.03,8]undec-5-yl 1H-indole-3-carboxylate], palonosetron [(3aS)-2-[(3S)-1-Azabicyclo[2.2.2]oct-3-yl]-2,3,3a,4,5,6-hexahydro-1H-benz[de]isoquinolin-1-one], ramosetron [(1-methyl-1H-indol-3-yl)[(5R)-4,5,6,7-tetrahydro-1H-benzimidazol-5-yl]methanone], alosetron [5-methyl-2-[(4-methyl-1H-imidazol-5-yl)methyl]-2,3,4,5-tetrahydro-1H-pyrido[4,3-b]indol-1-one], batanopride [4-amino-5-chloro-N-(2-diethylaminoethyl)-2-(3-oxobutan-2-yloxy)benzamide], renzapride [4-amino-N-[(4S,5S)-1-azabicyclo[3.3.1]non-4-yl]-5-chloro-2-methoxybenzamide] and zacopride [4-amino-5-chloro-2-methoxy-N-(quinuclidin-3-yl)benzamide].

According to an embodiment, the inhibition of HTR2A or HTR3 is performed together with activating β3-adrenergic receptor. As demonstrated in Examples, the activation of β3 adrenergic signaling simultaneously with inhibition of HTR2A or HTR3 is effective approach to enhanced energy expenditure in a white adipose tissue (WAT) and HTR3A is present in a brown adipose tissue (BAT), thereby preventing or treating metabolic diseases, e.g., obesity.

More specifically, the activation of β3-adrenergic receptor is performed by administering to the subject an agonist for β3-adrenergic receptor.

A multitude of agonists for β3-adrenergic receptor are well known to one of skill in the art.

According to an embodiment, the agonist for β3-adrenergic receptor is CL-316243 [5-[(2R)-2-[[(2R)-2-(3-Chlorophenyl)-2-hydroxyethyl]amino]propyl]-1,3-benzodioxole-2,2-dicarboxylic acid], amibegron [Ethyl ([(7S)-7-([(2R)-2-(3-chlorophenyl)-2-hydroxyethyl]amino)-5,6,7,8-tetrahydronaphthalen-2-yl]oxy)acetate], mirabegron [2-(2-Amino-1,3-thiazol-4-yl)-N-[4-(2-{[(2R)-2-hydroxy-2-phenylethyl]amino}ethyl)phenyl]acetamide], solabegron [3′-[(2-{[(2R)-2-(3-chlorophenyl)-2-hydroxyethyl]amino}ethyl)amino]biphenyl-3-carboxylic acid], L-742,791 [N-[4-[2-[[(2S)-2-hydroxy-3-(4-hydroxyphenoxy)propyl]amino]ethyl]phenyl]-4-iodobenzenesulfonamide] and L-796,568 [(R)—N-[4-[2-[[2-hydroxy-2-(3-pyridinyl)ethyl]amino]ethyl]-phenyl]-4-[4-[4-(trifluoromethyl)phenyl]thiazol-2-yl]-benzenesulfonamide, dihydrochloride].

According to an embodiment, the inhibition of HTR2A or HTR3 is performed by administering to the subject an antibody to HTR2A or HTR3A.

The antibody that may be used in the present invention is a polyclonal or monoclonal antibody, specifically a monoclonal antibody, that specifically binds to the Htr2a or Htr3a and inhibits its signaling. The antibody to the HTR2A or HTR3A protein may be prepared according to methods commonly employed in the art, for example, fusion method (Kohler and Milstein, European Journal of Immunology, 6: 511-519 (1976)), recombinant DNA method (U.S. Pat. No. 4,816,567) or phage antibody library method (Clackson et al, Nature, 352: 624-628 (1991); Marks et al, J. Mol. Biol., 222: 58, 1-597 (1991)). General procedures for producing antibody are described in detail in Harlow, E. and Lane, D., Using Antibodies: A Laboratory Manual, Cold Spring Harbor Press, New York, 1999; Zola, H., Monoclonal Antibodies: A Manual of Techniques, CRC Press, Inc., Boca Raton, Fla., 1984; and Coligan, Current Protocols in Immunology, Wiley/Greene, N Y, 1991, which are incorporated herein by references. For example, the preparation of hybridoma cells for monoclonal antibody production may be done by fusion of an immortal cell line and the antibody-producing lymphocytes, which can be achieved easily by techniques well known in the art. Polyclonal antibodies may be prepared by injecting HTR2A or HTR3A antigen to a suitable animal, collecting antiserum from the animal, and isolating antibodies employing a known affinity technique.

According to an embodiment, the inhibition of HTR2A or HTR3 is performed by administering to the subject a natural extract as antagonists against HTR2A or HTR3A.

As used herein, the term “natural extract” refers to an extract obtained from various organs or parts (e.g., leaves, flowers, roots, stems, branches, peel, fruits, etc.) of a natural source. The natural extract may be obtained using (a) water, (b) C1-C4 absolute or hydrated alcohol (e.g., methanol, ethanol, propanol, butanol, n-propanol, isopropanol, n-butanol, etc.), (c) mixture of the lower alcohol with water, (d) acetone, (e) ethyl acetate, (f) chloroform, (g) 1,3-butylene glycol, (h) hexane, or (i) diethyl ether as an extraction solvent.

Further, the natural extract includes, in addition to those obtained from solvent extraction, ones produced by common purification processes. For example, the natural extract includes the fractions obtained through various additional purification processes, such as separation using an ultrafiltration membrane having a predetermined molecular weight cut off, separation by various chromatographic techniques (based on size, charge, hydrophobicity or affinity). The natural extract may be prepared into powder through additional processes such as vacuum distillation, lyophilization or spray drying.

According to an embodiment of the present invention, the inhibition of HTR2A or HTR3 is performed by administering to the subject a peptide as antagonists against HTR2A or HTR3A.

As used herein, the term “peptide” refers to a straight-chain molecule consisting of amino acid residues linked by peptide bonds. It may consist of 4-40, specifically 4-30, most specifically 4-20, amino acid residues.

The HTR2A or HTR3A inhibitor peptide of the present invention is prepared according to the solid-phase synthesis technique commonly employed in the art (Merrifield, R. B., J. Am. Chem. Soc., 85: 2149-2154 (1963), Kaiser, E., Colescot, R. L., Bossinger, C. D., Cook, P. I., Anal. Biochem., 34: 595-598 (1970)). That is to say, amino acids with α-amino and side-chain groups protected are attached to a resin. Then, after removing the α-amino protecting groups, the amino acids are successively coupled to obtain an intermediate. The amino acid sequence for preparing the Htr2a or Htr3a inhibitor peptide of the present disclosure may be referred to in the existing techniques (Chen L, Hahn H, Wu G, Chen C H, Liron T, Schechtman D, Cavallaro G, Banci L, Guo Y, Bolli R, Dorn G W, Mochly-Rosen D., Proc. Natl. Acad. Sci., 98, 11114-9 (2001); Phillipson A, Peterman E E, Taormina P Jr, Harvey M, Brue R J, Atkinson N, Omiyi D, Chukwu U, Young L H., Am. J. Physiol. Heart Circ. Physiol., 289, 898-907 (2005); and Wang J, Bright R, Mochly-Rosen D, Giffard R G., Neuropharmacology., 47, 136-145 (2004)).

According to an embodiment of the present invention, the inhibition of HTR2A or HTR3 is performed by administering to the subject an aptamer as antagonists against HTR2A or HTR3A. Aptamers may be oligo nucleic acid (e.g., RNA) or peptide molecules, of which details can be found in Bock L C et al., Nature 355 (6360):5646 (1992); Hoppe-Seyler F, Butz K “Peptide aptamers: powerful new tools for molecular medicine”. J Mol Med. 78(8):42630(2000); Cohen B A, Colas P, Brent R. “An artificial cell-cycle inhibitor isolated from a combinatorial library”. Proc Natl Acad Sci USA. 95(24):142727 (1998).

The inhibition of HTR2A or HTR3 in the subject may be performed by administering to the subject a composition containing the active ingredient described above.

The composition of the present invention may be prepared as a pharmaceutical composition or a food composition.

Where the composition of the present invention is a pharmaceutical composition, the composition includes: (i) an effective amount of the HTR2A or HTR3 inhibitor; and (ii) a pharmaceutically acceptable carrier. As used herein, the term “effective amount” means an amount sufficient to exert the therapeutic effects described herein.

The pharmaceutically acceptable carrier included in the pharmaceutical composition of the present invention is one commonly used in the art and includes carbohydrate compounds (e.g., lactose, amylose, dextrose, sucrose, sorbitol, mannitol, starch and cellulose), gum acacia, calcium phosphate, alginate, gelatin, calcium silicate, microcrystalline cellulose, polyvinylpyrrolidone, cellulose, water, syrup, salt solution, alcohol, gum arabic, vegetable oils (e.g., corn oil, cottonseed oil, soybean oil, olive oil, or coconut oil), polyethylene glycol, methyl cellulose, methyl hydroxybenzoate, propyl hydroxybenzoate, talc, magnesium stearate, mineral oil, etc., but is not limited thereto. The pharmaceutical composition of the present invention may further include, in addition to the above ingredients, a lubricant, a wetting agent, a sweetener, a flavor, an emulsifier, a suspending agent, a preservative, or the like. Suitable pharmaceutically acceptable carriers and preparations are described in detail in Remington's Pharmaceutical Sciences (19th ed., 1995).

The pharmaceutical composition of the present disclosure may be administered orally or parenterally. Methods for parenteral administration include intravenous injection, subcutaneous injection, intramuscular injection and the like.

An adequate administration dose of the pharmaceutical composition of the present invention may vary depending on various factors, such as method of preparation, method of administration, age, body weight, sex and physical conditions of the patient, diet, administration period, administration route, excretion rate, and response sensitivity. A physician of ordinary skill in the art will easily determine and diagnose an administration dose effective for the desired treatment or prevention. In a specific embodiment of the present invention, the adequate administration dose is 0.0001-100 mg/kg (body weight) per day. The administration can be given once or several times a day.

The pharmaceutical composition of the present invention may be formulated into a unit or multiple dosage form using a pharmaceutically acceptable carrier and/or excipient according to a method commonly known in the art. The formulation may be a solution in an oily or aqueous medium, a suspension or emulsion, an extract, a powder, a granule, a tablet, or a capsule. It may further include a dispersant or a stabilizer.

The subject to be prevented or treated includes mammalians, particularly human.

The composition of the present invention may be prepared as a food composition, particularly a functional food composition. The functional food composition of the present invention includes ingredients commonly used in the preparation of food. For example, it may include proteins, carbohydrates, fats, nutrients and flavoring agents. For instance, a drink may further include, in addition to the Htr2a Htr3a inhibitor as the active ingredient, a flavoring agent or a natural carbohydrate. For example, the natural carbohydrate may be a monosaccharide (e.g., glucose, fructose, etc.), a disaccharide (e.g., maltose, sucrose, etc.), an oligosaccharide, a polysaccharide (e.g., dextrin, cyclodextrin, etc.), or a sugar alcohol (e.g., xylitol, sorbitol, erythritol, etc.). The flavoring agent may be a natural flavoring agent (e.g., thaumatin, stevia extract, etc.) or a synthetic flavoring agent (e.g., saccharin, aspartame, etc.).

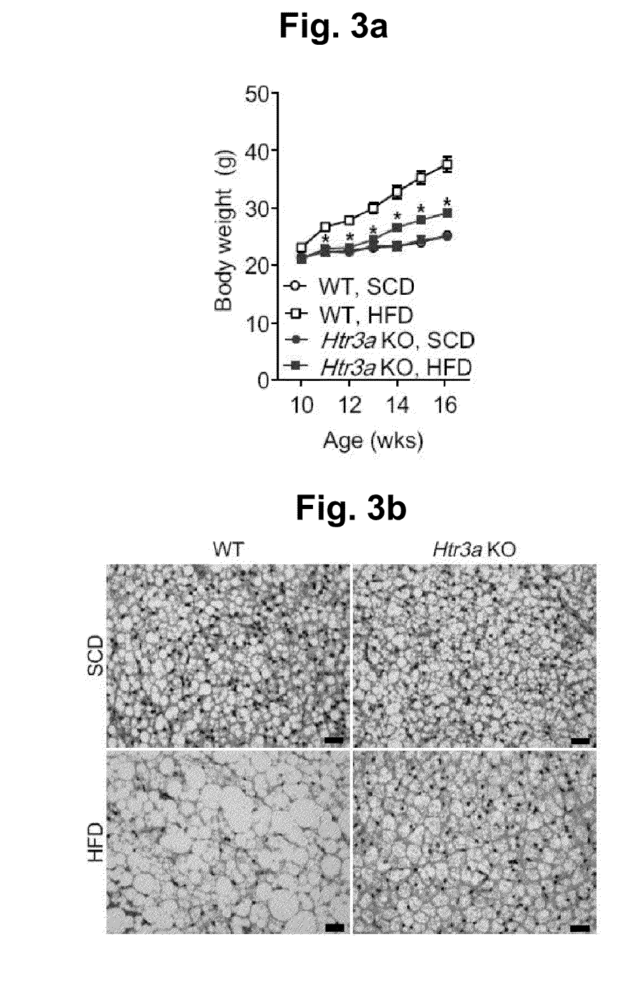

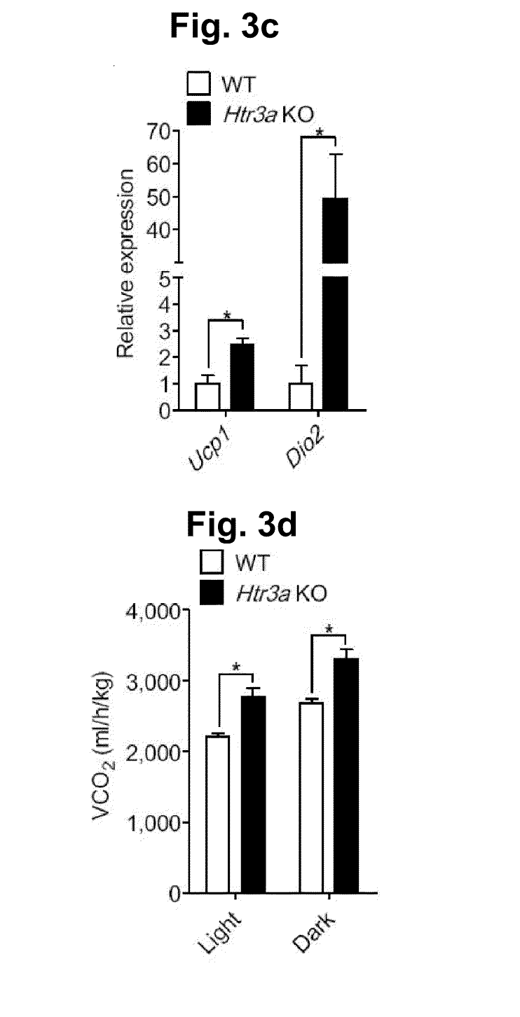

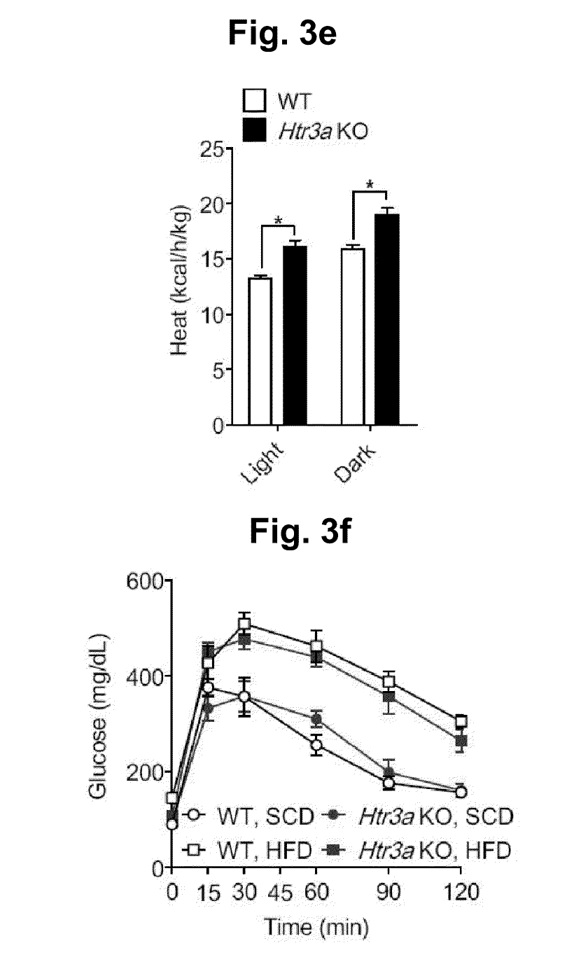

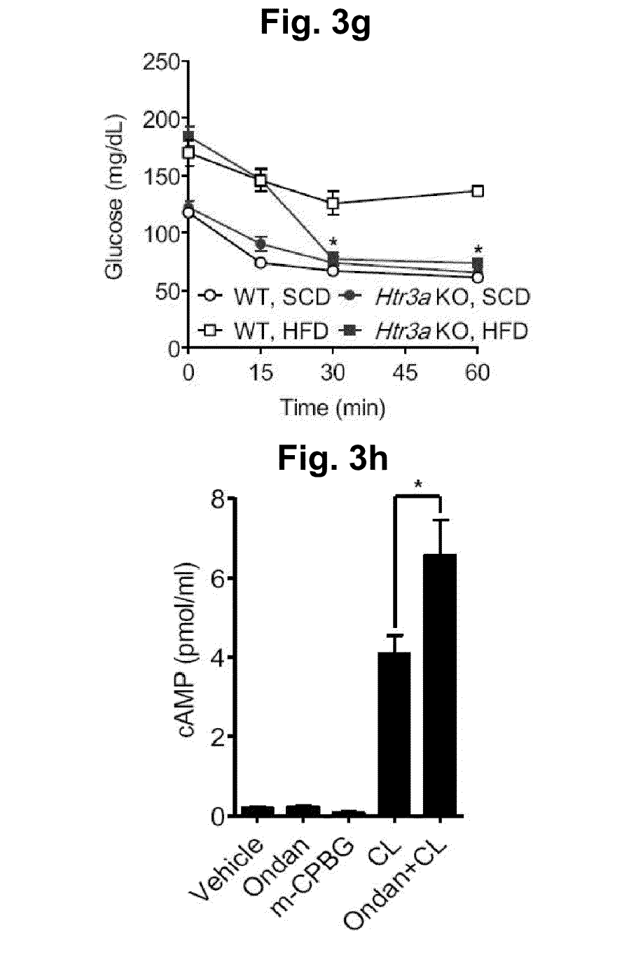

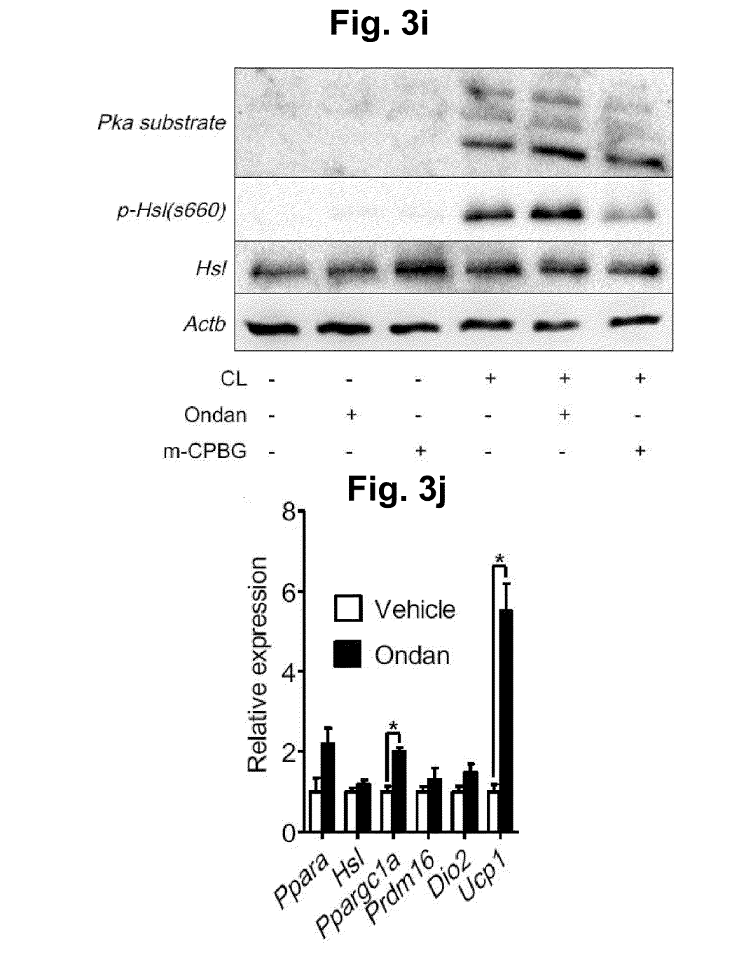

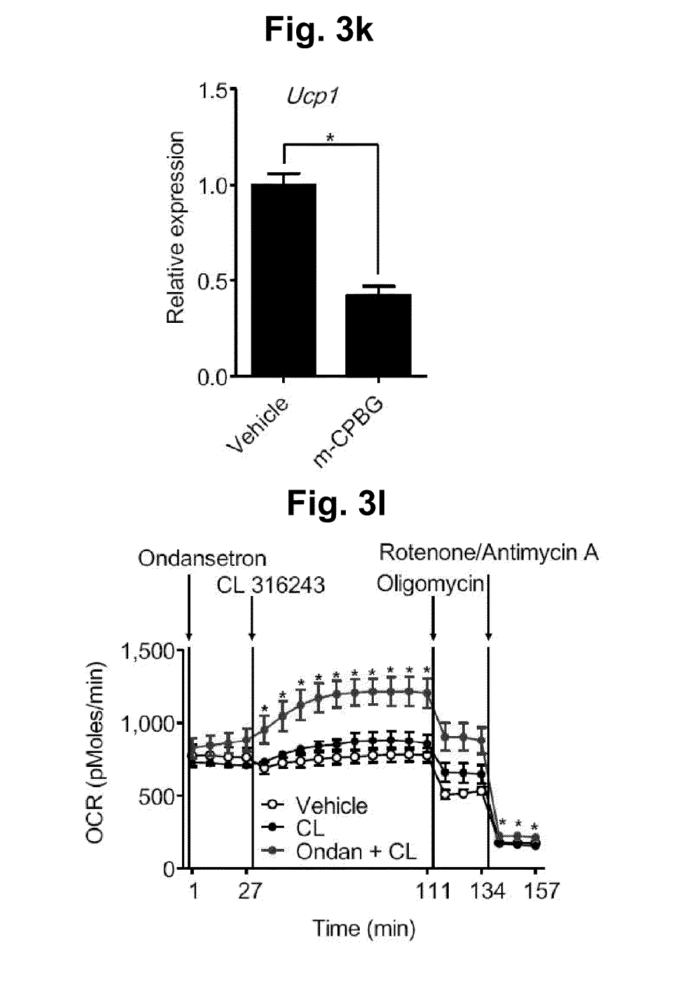

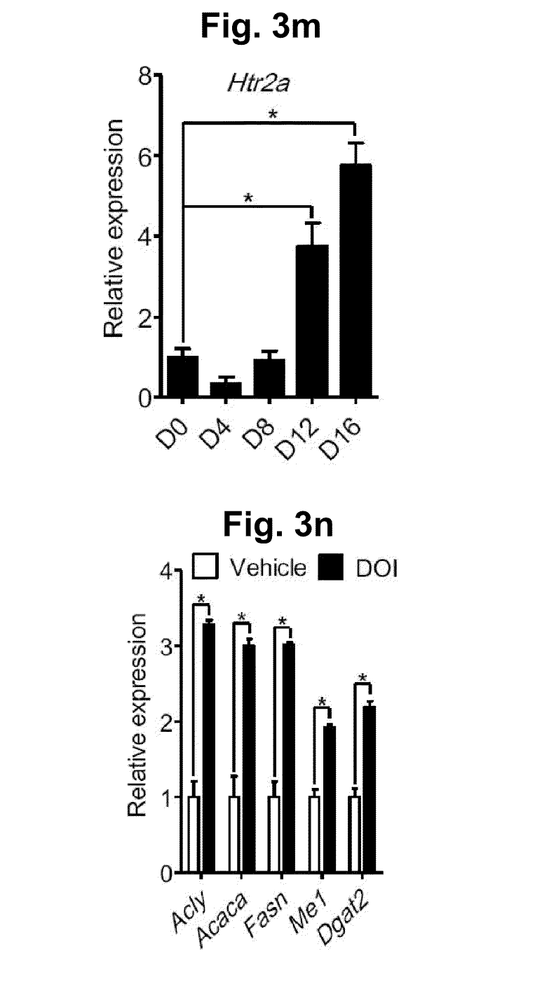

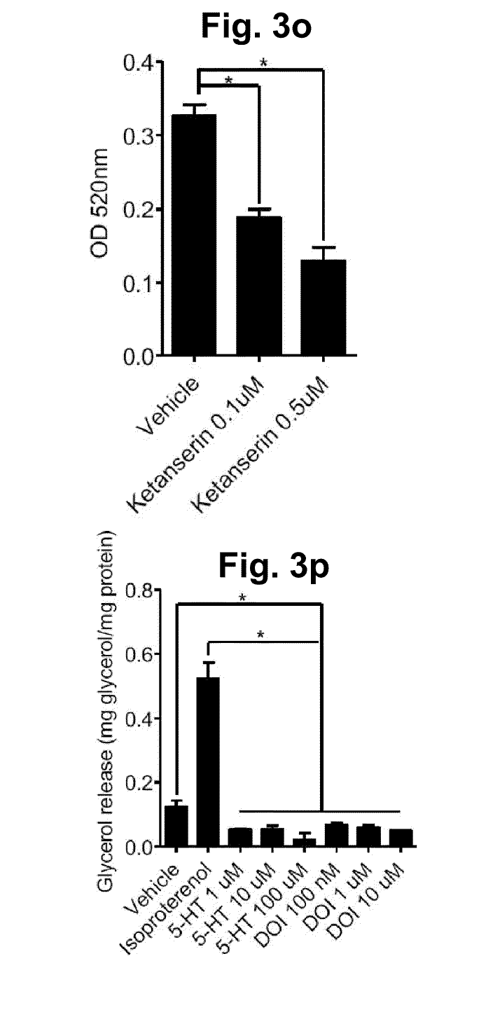

According to an embodiment, HTR2A to be inhibited is present in a white adipose tissue (WAT) or white adipose cell, and HTR3A is present in a brown adipose tissue (BAT) or brown adipose cell. As addressed in Examples and FIGS. 3a-3p, the inhibition of Htr2a in the WAT contributes to increase in lipolysis and browning, and the inhibition of Htr3a in the BAT contributes to increase in thermogenesis.

The metabolic diseases to be prevented or treated by the present invention include obesity, diabetes, insulin resistance, hyperlipidemia or hypercholesterolemia. More particularly, the metabolic disease to be prevented or treated by the present invention is obesity.

As demonstrated in Examples and FIGS. 3a-3p, the inhibition of Htr3a (e.g., knock-out of the Htr3a gene and treatment of Htr3a antagonist) contributes to resistance to obesity. In addition, the inhibition of Htr3a results in decrease of lipid droplet size in the BAT, increase of expression levels of thermogenic genes (e.g., Ucp1 and Dio2) in the BAT (FIGS. 3c and 3k), increase of carbon dioxide production (VCO₂) (FIG. 3d) and heat production (FIG. 3e) and increase of insulin sensitivity (FIG. 3g). Interestingly, the inhibition of Htr3a increases cAMP levels and phosphorylation of PKA pathway in the BAT only in the presence of a β3-adrenergic receptor agonist (FIGS. 3h, 3i, 3j), and the oxygen consumption rate in the BAT is increased by the inhibition of Htr3a along with a β3-adrenergic receptor agonist in a synergistic manner (FIG. 3l). Furthermore, the inhibition of Htr3a permits to decrease LDL cholesterol and leptin and increase free fatty acid level (FIG. 3r). The inhibition of Htr3a leads to increased size and number of mitochondria in the BAT (FIG. 3t). As demonstrated in Examples and FIGS. 3m, 3n, 3o, Htr2a is shown to be involved in lipogenesis.

In summary, the inhibition of HTR2A or HTR3 is a promising strategy to prevent or treat metabolic diseases including obesity, diabetes, insulin resistance, hyperlipidemia or hypercholesterolemia, particularly, obesity.

In another aspect of this invention, there is provided a method for screening a therapeutic agent for treating a metabolic disease, comprising:

(a) contacting a test substance of interest for analysis to HTR2A (5-hydroxy tryptamine 2a receptor) or HTR3A (5-hydroxy tryptamine 3a receptor); and

(b) analyzing whether the test substance inhibits HTR2A or HTR3A;

wherein where the test substance inhibits HTR2A or HTR3A, it is determined as the therapeutic agent for treating the metabolic disease.

According to the present method, HTR2A or HTR3A is first contacted to a test substance to be analyzed.

In the present invention, HTR2A or HTR3A may be in an isolated or purified form or in a cell. According to an embodiment, HTR2A is present in a white adipose cell and HTR3A is present in a brown adipose cell.

The term “test substance” used herein in conjunction with the present screening method refers to a material tested in the present method for analyzing the influence on HTR2A or HTR3A. The test substance includes siRNA, antisense oligonucleotides, antibodies, aptamers, extracts of natural sources and chemical substances. The test substance to be analyzed by the screening method of the present disclosure may be an individual compound or a mixture of compounds (e.g., natural extract, or cell or tissue culture). The test substance may be obtained from a library of synthetic or natural compounds. The method for obtaining the library of such compounds is known in the art. A library of synthetic compounds is commercially available from Maybridge Chemical Co. (UK), Comgenex (USA), Brandon Associates (USA), Microsource (USA) and Sigma-Aldrich (USA), and a library of natural compounds is commercially available from Pan Laboratories (USA) and MycoSearch (USA). The test substance may be obtained through various known combinational library methods. For example, it may be acquired by a biological library method, a spatially-addressable parallel solid phase or solution phase library method, a synthetic library method requiring deconvolution, a “one-bead/one-compound” library method, and a synthetic library method using affinity chromatography selection. The methods for obtaining the molecular libraries are described in DeWitt et al., Proc. Natl. Acad. Sci. U.S.A. 90, 6909, 1993; Erb et al. Proc. Natl. Acad. Sci. U.S.A. 91, 11422, 1994; Zuckermann et al., J. Med. Chem. 37, 2678, 1994; Cho et al., Science 261, 1303, 1993; Carell et al., Angew. Chem. Int. Ed. Engl. 33, 2059, 1994; Carell et al., Angew. Chem. Int. Ed. Engl. 33, 2061; Gallop et al., J. Med. Chem. 37, 1233, 1994, and so forth.

The screening method of the present disclosure may be carried out variously. Particularly, it may be performed in a high-throughput manner using various known binding assay, expression assay or activity assay techniques.

According to an embodiment, the inhibition of HTR2A or HTR3A is suppression of the expression of HTR2A or HTR3A.

The expression of HTR2A or HTR3A may be performed by measuring the expression of the HTR2A or HTR3A gene. The measurement of the expression of the Htr2a or Htr3a gene may be carried out by various methods known to those ordinarily skilled in the art, for example, RT-PCR (Sambrook et al., Molecular Cloning. A Laboratory Manual, 3rd ed. Cold Spring Harbor Press (2001)), Northern blotting (Peter B. Kaufma et al., Molecular and Cellular Methods in Biology and Medicine, 102-108, CRC press), cDNA microarray hybridization (Sambrook et al., Molecular Cloning. A Laboratory Manual, 3rd ed. Cold Spring Harbor Press (2001)) or in situ hybridization (Sambrook et al., Molecular Cloning. A Laboratory Manual, 3rd ed. Cold Spring Harbor Press (2001)).

The expression of HTR2A or HTR3A may be performed by measuring the expression of the HTR2A or HTR3A protein. The measurement of the expression of the HTR2A or HTR3A protein may be carried out by various methods known to those ordinarily skilled in the art, for example, Western blotting, radioactivity immunoanalysis, radioactive immunoprecipitation, immunoprecipitation, enzyme-linked immunosorbent assay (ELISA), capture-ELISA, inhibition or competition assay or sandwich immunoanalysis.

Alternatively, the analysis of whether the test substance inhibits HTR2A or HTR3A is to analyze suppression of function of HTR2A or HTR3A.

According to an embodiment, the contacting of the test substance to HTR3A in the BAT is performed together with activating β3-adrenergic receptor.

According to an embodiment, the metabolic disease is obesity, diabetes, insulin resistance, hyperlipidemia or hypercholesterolemia.

The features and advantages of the present invention will be summarized as follows:

(a) The present invention is drawn to a novel approach to prevent or treat metabolic diseases including obesity, diabetes, insulin resistance, hyperlipidemia or hypercholesterolemia, particularly, obesity.

(b) The present invention suggests HTR2A and HTR3A, particularly HTR3A as a promising therapeutic target for metabolic diseases, particularly, obesity.

(c) The inhibition of HTR3A leads to various advantageous efficacies for therapy of metabolic diseases, including resistance to obesity, decrease of lipid droplet size, increase of expression levels of thermogenic genes, increase of metabolic activities, increase of insulin sensitivity and decrease of LDL cholesterol and leptin.

(d) Interestingly, the inhibition of HTR3A increases cAMP levels and phosphorylation of PKA pathway in the BAT only in the presence of a β3-adrenergic receptor agonist, and the oxygen consumption rate in the BAT is increased by the inhibition of HTR3A along with a β3-adrenergic receptor agonist in a synergistic manner, demonstrating that co-administration of HTR3A antagonists and β3-adrenergic receptor agonists is a dramatic therapy for metabolic diseases, particularly, obesity.

The present invention will now be described in further detail by examples. It would be obvious to those skilled in the art that these examples are intended to be more concretely illustrative and the scope of the present invention as set forth in the appended claims is not limited to or by the examples.

### EXAMPLES

### Methods

Reagents.

PCPA, D-glucose, insulin, CL-316243 (β3-adrenergic receptor agonist), ondansetron, m-CPBG, triiodothyronine (T3), 3-isobutyl-1-methylxanthine (IBMX), indomethacin, dexamethasone, Oil Red 0 dye, ketanserin, DOI, isopropyl alcohol (IPA), formalin, ascorbic acid, perchloric acid, tamoxifen and polyethylene glycol 400 (PEG-400) were purchased from Sigma (St. Louis, Mo., USA). LP-533401 was purchased from Dalton Pharma Services (Toronto, Ontario, Canada). TRIzol reagent, Dulbecco's modified Eagle's medium (DMEM), calf serum, fetal bovine serum (FBS), and penicillin-streptomycin (P/S) were obtained from Invitrogen (Carlsbad, Calif., USA). All antibodies were purchased from Cell Signaling Technologies (Denvers, Mass., USA) unless otherwise specified.

Animals and Diets.

The generation of Tph^{flox/flox} mice, Adipoq-cre mice, and aP2-CreERT2 mice has previously been reported^{17,30,31}. C57BL/6J mice, Htr3a-targeted KO mice (B6.129X1-Htr3a^{tm1jul/J}, Htr3a KO) and leptin-deficient ob/ob mice (B6.V-Lep^{ob}/J) were purchased from the Jackson Laboratory (Bar Harbor, Me., USA). To create the adipose tissue-specific Tph1 KO mice, Tph1^{flox/flox} mice were crossed with Adipoq-Cre mice (Tph1 FKO mice) and aP2-CreERT2 mice (Tph1 AFKO mice). The mice were housed in climate-controlled, specific pathogen-free (SPF) barrier facilities under a 12-h light-dark cycle, and chow and water were provided ad libitum. The Institutional Animal Care and Use Committee at the Korea Advanced Institute of Science and Technology approved the experimental protocols for this study. Htr3a KO mice and Tph1^{flox/flox} mice were backcrossed with C57BL/6J mice for more than 10 generations. Cre-recombination of 6-week-old Tph1 AFKO mice was induced by intraperitoneal (IP) injection of 5 doses of 2 mg of tamoxifen (Sigma) for 1 week. We fed male mice (aged 8˜10 weeks) either on SCD (standard chow diet; 12% fat calories, Purina Laboratory Rodent Diets 38057) or HFD (60% fat calories, Research Diets D12492). PBS or 300 mg/kg PCPA was administered as a daily intraperitoneal injection. LP-533401 was dissolved in PEG-400 and 5% dextrose (40:60 ratio). Vehicle or 30 mg/kg LP-533401 was administered daily with a feeding needle. We randomly divided C57BL/6J mice into 2-4 groups. For transgenic mice, we compared data between KO mice and their WT littermates. No blinding was done.

Cell culture.

Murine 3T3-L1 cells (American Type Culture Collection) were cultured in DMEM supplemented with 10% fetal calf serum and 100 μg/mL P/S in a humidified atmosphere of 5% CO₂ at 37° C. Two days after reaching confluence, the cells were induced to differentiate using medium supplemented with 0.5 mM IBMX, 1 mg/mL insulin and 1 μM dexamethasone (day 0). After two days, the medium was replaced with DMEM supplemented with 1 mg/mL insulin in 10% FBS and P/S (day 2), and it was changed every two days from days 4 to 8.

IBAs were cultured in DMEM supplemented with 10% FBS and P/S in a humidified atmosphere of 5% CO₂ at 37° C.³². After reaching 95% confluence, the cells were induced to differentiate using DMEM with 10% FBS, 0.5 μg/mL insulin, 1 nM T3, 0.125 mM indomethacin, 2 μg/mL dexamethasone, 0.5 μM IBMX and P/S (day 0). After two days, the medium was replaced with DMEM supplemented with 10% FBS, 0.5 μg/mL insulin, 1 nM T3 and P/S (day 2), and it was changed every two days from days 4 to 8. We confirmed that these cell lines are free from Mycoplasma infection.

SVF Isolation.

The stromal vascular fraction of epididymal, inguinal and BAT from 6- to 7-week-old mice was separated by collagenase digestion. Briefly, the adipose tissues were dissected, minced and digested with 0.2% collagenase A (Roche) in Hank's balanced salts solution (Sigma) for 45 min at 37° C. with constant shaking. Mature adipocytes and connective tissues were separated from the cell pellet by centrifugation at 800×g for 10 min at 4° C. The cell pellet was then suspended with RBC lysis buffer (Sigma) and filtered through a 40-μm mesh filter (BD bioscience). The pelleted stromal vascular cells (SVCs) were re-suspended in DMEM containing 10% FBS and seeded in 6-well plates for adipogenic differentiation.

Oil Red O Staining.

After full differentiation (day 8), 3T3-L1 adipocytes were fixed with 3.7% (w/v) formaldehyde in PBS for 15 min at room temperature and then washed three times with PBS. Then, the cells were stained with filtered Oil Red 0 solution (1.5 mg/mL 60% (v/v) isopropanol) for 30 min and then rinsed twice with distilled water. To quantify the amount of Oil Red 0 staining, the cells were eluted with 100% isopropranol for 10 min, and the absorbance densities of the extracts were measured at 520 nm using a VersaMax microplate reader (Molecular Devices, Sunnyvale, Calif., USA).

Metabolic Analysis.

To measure the metabolic rate, the mice were housed individually in an eight-chamber, open-circuit Oxymax/CLAMS (Columbus Instruments Comprehensive Lab Animal Monitoring System) system as previously described³³. Each mouse was assessed for 72 h in the fed state to determine their metabolic rates. PET imaging was performed using a microPET R4 scanner (Concorde Microsystems, Siemens) as previously described³⁴.

Glucose Tolerance Test and Insulin Tolerance Test.

For the glucose tolerance tests, the mice were administered 2 g/kg D-glucose in PBS after overnight fasting. For the insulin tolerance tests, the mice were intraperitoneally injected with insulin (0.75 U/kg) after being fasted for 4 h. The blood glucose levels in blood samples obtained from tail veins 0, 15, 30, 45, 60, 90 and 120 min after injection were measured using a Gluco DR Plus glucometer (Allmedicus).

Blood Chemistry Analysis.

Tissue serotonin was extracted by homogenization in extraction buffer containing 0.02% ascorbic acid in 0.1 M perchloric acid followed by centrifugation. The serotonin levels in the supernatants were measured via the liquid chromatography-mass spectrometry method. Serum leptin (Enzo Life Science, Farmingdale, N.Y., USA), LDL-cholesterol (Waco), and FFA (Biovision, Mountain View, Calif., USA) were measured using enzyme-linked immunosorbent assay (ELISA) kits.

cAMP Assay.

Differentiated IBAs were treated with 1 uM ondansetron or 100 nM m-CPBG or 1 uM CL-316243 for 15 min. 1 uM CL-315243 was used as a positive control. The cAMP competitive ELISA (Promega) was performed according to the manufacturer's instruction. Briefly, cAMP was extracted by adding 0.1 M HCl with 0.5% triton X-100 to the cells. After centrifugation at 600 g for 10 min, the supernatant was used for the determination of cAMP levels by competitive cAMP ELISA.

OCR Assay.

The OCRs of the cells were measured using a Seahorse XF analyzer (Seahorse Bioscience, Billerica, Mass., USA). After seeding IBAs on an XF-24 plate, the cells were incubated and differentiated using the protocol described above. Fully differentiated IBAs were pre-treated with PBS and ondansetron for 30 min. Then, the IBAs were treated with the β3 agonist (CL-316243) and the mitochondrial inhibitors oligomycin and rotenone/antimycin. The OCRs were calculated and recorded by a sensor cartridge and the Seahorse XF-24 software.

Real-Time PCR Analysis.

Total RNA was extracted from the mouse tissues or cell lines using TRIzol reagent (Invitrogen) according to the manufacturer's protocol. After TURBO DNase (Invitrogen) treatment, 2 μg of total RNA was used to generate complementary DNA with Superscript III reverse transcriptase (Invitrogen). To analyze gene expression, real-time PCR was performed with a ViiA 7 Real-Time PCR system (Applied Biosystems) and the Power SYBR Green PCR master mix (Applied Biosystems). Relative quantification was based on the ddCt method, and ActB was used as an endogenous control (internal control). The primer sequences are provided in Table 1.

TABLE 1

| Target | Primer | Sequence | SEQ ID number |
|---|---|---|---|
| Acaca | Forward Primer (5′-3′) | CAGTAACCTGGTGAAGCTGGA | SEQ ID NO: 1 |
| Acaca | Reverse Primer (5′-3′) | GCCAGACATGCTGGATCTCAT | SEQ ID NO: 2 |
| Acly | Forward Primer (5′-3′) | CCCTCTTCAGCCGACATACC | SEQ ID NO: 3 |
| Acly | Reverse Primer (5′-3′) | CTGCTTGTGATCCCCAGTGA | SEQ ID NO: 4 |
| Actb | Forward Primer (5′-3′) | CAGCTTCTTTGCAGCTCCTT | SEQ ID NO: 5 |
| Actb | Reverse Primer (5′-3′) | CTTCTCCATGTCGTCCCAGT | SEQ ID NO: 6 |
| Adipoq | Forward Primer (5′-3′) | CTCCACCCAAGGGAACTTGT | SEQ ID NO: 7 |
| Adipoq | Reverse Primer (5′-3′) | GGACCAAGAAGACCTGCATC | SEQ ID NO: 8 |
| Cidea | Forward Primer (5′-3′) | GCCGTGTTAAGGAATCTGCTG | SEQ ID NO: 9 |
| Cidea | Reverse Primer (5′-3′) | TGCTCTTCTGTATCGCCCAGT | SEQ ID NO: 10 |
| Cox8b | Forward Primer (5′-3′) | GAACCATGAAGCCAACGACT | SEQ ID NO: 11 |
| Cox8b | Reverse Primer (5′-3′) | GCGAAGTTCACAGTGGTTCC | SEQ ID NO: 12 |
| Cptla | Forward Primer (5′-3′) | AGCTCGCACATTACAAGGACA | SEQ ID NO: 13 |
| Cptla | Reverse Primer (5′-3′) | CCAGCACAAAGTTGCAGGAC | SEQ ID NO: 14 |
| Cycs | Forward Primer (5′-3′) | GCAAGCATAAGACTGGACCAAA | SEQ ID NO: 15 |
| Cycs | Reverse Primer (5′-3′) | TTGTTGGCATCTGTGTAAGAGAATC | SEQ ID NO: 16 |
| Dgat1 | Forward Primer (5′-3′) | GGATCTGAGGTGCCATCGTC | SEQ ID NO: 17 |
| Dgat1 | Reverse Primer (5′-3′) | ATCAGCATCACCACACACCA | SEQ ID NO: 18 |
| Dgat2 | Forward Primer (5′-3′) | CATCATCGTGGTGGGAGGTG | SEQ ID NO: 19 |
| Dgat2 | Reverse Primer (5′-3′) | TGGGAACCAGATCAGCTCCAT | SEQ ID NO: 20 |
| Dio2 | Forward Primer (5′-3′) | TTGGGGTAGGGAATGTTGGC | SEQ ID NO: 21 |
| Dio2 | Reverse Primer (5′-3′) | TCCGTTTCCTCTTTCCGGTG | SEQ ID NO: 22 |
| Fabp4 | Forward Primer (5′-3′) | AACACCGAGATTTCCTTCAA | SEQ ID NO: 23 |
| Fabp4 | Reverse Primer (5′-3′) | TCACGCCTTTCATAACACAT | SEQ ID NO: 24 |
| Fasn | Forward Primer (5′-3′) | AAGCGGTCTGGAAAGCTGAA | SEQ ID NO: 25 |
| Fasn | Reverse Primer (5′-3′) | AGGCTGGGTTGATACCTCCA | SEQ ID NO: 26 |
| Gpam | Forward Primer (5′-3′) | CCACAGAGCTGGGAAAGGTT | SEQ ID NO: 27 |
| Gpam | Reverse Primer (5′-3′) | GTGCCTTGTGTGCGTTTCAT | SEQ ID NO: 28 |
| Hsl | Forward Primer (5′-3′) | AACGAGACAGGCCTCAGTGT | SEQ ID NO: 29 |
| Hsl | Reverse Primer (5′-3′) | GAATCGGCCACCGGTAAAGA | SEQ ID NO: 30 |
| Htr1a | Forward Primer (5′-3′) | TCAGCTACCAAGTGATCACCTCT | SEQ ID NO: 31 |
| Htr1a | Reverse Primer (5′-3′) | GTCCACTTGTTGAGCACCTG | SEQ ID NO: 32 |
| Htr1b | Forward Primer (5′-3′) | TGCTCCTCATCGCCCTCTATG | SEQ ID NO: 33 |
| Htr1b | Reverse Primer (5′-3′) | CTAGCGGCCATGAGTTTCTTCTT | SEQ ID NO: 34 |
| Htr1d | Forward Primer (5′-3′) | CCTCCAACAGATCCCTGAATG | SEQ ID NO: 35 |
| Htr1d | Reverse Primer (5′-3′) | CAGAGCAATGACACAGAGATGCA | SEQ ID NO: 36 |
| Htr1f | Forward Primer (5′-3′) | TGTGAGAGAGAGCTGGATTATGG | SEQ ID NO: 37 |
| Htr1f | Reverse Primer (5′-3′) | TAGTTCCTTGGTGCCTCCAGAA | SEQ ID NO: 38 |
| Htr2a | Forward Primer (5′-3′) | AGCTGCAGAATGCCACCAACTAT | SEQ ID NO: 39 |
| Htr2a | Reverse Primer (5′-3′) | GGGATTGGCATGGATATACCTAC | SEQ ID NO: 40 |
| Htr2b | Forward Primer (5′-3′) | AAATAAGCCACCTCAACGCCT | SEQ ID NO: 41 |
| Htr2b | Reverse Primer (5′-3′) | TCCCGAAATGTCTTATTGAAGAG | SEQ ID NO: 42 |
| Htr2c | Forward Primer (5′-3′) | TTCTTAATGTCCCTAGCCATTGC | SEQ ID NO: 43 |
| Htr2c | Reverse Primer (5′-3′) | GCAATCTTCATGATGGCCTTAGT | SEQ ID NO: 44 |
| Htr3a | Forward Primer (5′-3′) | AAATCAGGGCGAGTGGGAGCTG | SEQ ID NO: 45 |
| Htr3a | Reverse Primer (5′-3′) | GACACGATGATGAGGAAGACTG | SEQ ID NO: 46 |
| Htr3b | Forward Primer (5′-3′) | CGTGTGGTACCGAGAGGTTT | SEQ ID NO: 47 |
| Htr3b | Reverse Primer (5′-3′) | GGATGGGCTTGTGGTTTCTA | SEQ ID NO: 48 |
| Htr4 | Forward Primer (5′-3′) | ATGGACAAACTTGATGCTAATGTGA | SEQ ID NO: 49 |
| Htr4 | Reverse Primer (5′-3′) | TCACCAGCACCGAAACCAGCA | SEQ ID NO: 50 |
| Htr5a | Forward Primer (5′-3′) | GATTGACTTCAGTGGGCTCG | SEQ ID NO: 51 |
| Htr5a | Reverse Primer (5′-3′) | AAAGTCAGGACTAGCACTCG | SEQ ID NO: 52 |
| Htr7 | Forward Primer (5′-3′) | CTCGGTGTGCTTTGTCAAGA | SEQ ID NO: 53. |
| Htr7 | Reverse Primer (5′-3′) | TTGGCCATACATTTCCCATT | SEQ ID NO: 54 |
| Lep | Forward Primer (5′-3′) | ACACACGCAGTCGGTATCC | SEQ ID NO: 55 |
| Lep | Reverse Primer (5′-3′) | GCAGCACATTTTGGGAAGGC | SEQ ID NO: 56 |
| Lpin1 | Forward Primer (5′-3′) | CATACAAAGGCAGCCACACG | SEQ ID NO: 57 |
| Lpin1 | Reverse Primer (5′-3′) | CATACAAAGGCAGCCACACG | SEQ ID NO: 58 |
| Maoa | Forward Primer (5′-3′) | GCGGTACAAGGGTCTGTTCC | SEQ ID NO: 59 |
| Maoa | Reverse Primer (5′-3′) | CAGCCAATCCTGAGATGCCG | SEQ ID NO: 60 |
| Maob | Forward Primer (5′-3′) | GGGCGGCATCTCAGGTATGG | SEQ ID NO: 61 |
| Maob | Reverse Primer (5′-3′) | AAGTCCTGCCTCCTACACGG | SEQ ID NO: 62 |
| Me1 | Forward Primer (5′-3′) | GACCCGCATCTCAACAAGGA | SEQ ID NO: 63 |
| Me1 | Reverse Primer (5′-3′) | CAGGAGATACCTGTCGAAGTCA | SEQ ID NO: 64 |
| Nrf1 | Forward Primer (5′-3′) | CAGCAACCCTGATGGCACCGTGTCG | SEQ ID NO: 65 |
| Nrf1 | Reverse Primer (5′-3′) | GGCCTCTGATGCTTGCGTCGTCTGG | SEQ ID NO: 66 |
| Plin1 | Forward Primer (5′-3′) | GGTGTTACAGCGTGGAGAGTA | SEQ ID NO: 67 |
| Plin1 | Reverse Primer (5′-3′) | TCTGGAAGCACTCACAGGTC | SEQ ID NO: 68 |
| Pparg | Forward Primer (5′-3′) | GGTGTGATCTTAACTGCCGGA | SEQ ID NO: 69 |
| Pparg | Reverse Primer (5′-3′) | GCCCAAACCTGATGGCATTG | SEQ ID NO: 70 |
| Ppargc1a | Forward Primer (5′-3′) | GCCCAGGTACGACAGCTATG | SEQ ID NO: 71 |
| Ppargc1a | Reverse Primer (5′-3′) | ACGGCGCTCTTCAATTGCTT | SEQ ID NO: 72 |
| Prdm16 | Forward Primer (5′-3′) | AGCCCTCGCCCACAACTTGC | SEQ ID NO: 73 |
| Prdm16 | Reverse Primer (5′-3′) | TGACCCCCGGCTTCCGTTCA | SEQ ID NO: 74 |
| Scd1 | Forward Primer (5′-3′) | AGAGTCAGGAGGGCAGGTTT | SEQ ID NO: 75 |
| Scd1 | Reverse Primer (5′-3′) | GAACTGGAGATCTCTTGGAGCA | SEQ ID NO: 76 |
| Slc6a4 | Forward Primer (5′-3′) | CGCAGTTCCCAGTACAAGC | SEQ ID NO: 77 |
| Slc6a4 | Reverse Primer (5′-3′) | CGTGAAGGAGGAGATGAGG | SEQ ID NO: 78 |
| Srebf1 | Forward Primer (5′-3′) | GTGGGCCTAGTCCGAAGC | SEQ ID NO: 79 |
| Srebf1 | Reverse Primer (5′-3′) | CTGGAGCATGTCTTCGATGT | SEQ ID NO: 80 |
| Tfam | Forward Primer (5′-3′) | AGTTCCCACGCTGGTAGTGT | SEQ ID NO: 81 |
| Tfam | Reverse Primer (5′-3′) | GCGCACATCTCGACCC | SEQ ID NO: 82 |
| Tmem26 | Forward Primer (5′-3′) | ACCCTGTCATCCCACAGAG | SEQ ID NO: 83 |
| Tmem26 | Reverse Primer (5′-3′) | TGTTTGGTGGAGTCCTAAGGTC | SEQ ID NO: 84 |
| Tph1 | Forward Primer (5′-3′) | ACCATGATTGAAGACAACAAGGAG | SEQ ID NO: 85 |
| Tph1 | Reverse Primer (5′-3′) | TCAACTGTTCTCGGCTGAT | SEQ ID NO: 86 |
| Tph2 | Forward Primer (5′-3′) | GCCATGCAGCCCGCAATGATGATG | SEQ ID NO: 87 |
| Tph2 | Reverse Primer (5′-3′) | CAACTGCTGTCTTGCTGCTC | SEQ ID NO: 88 |
| Ucp1 | Forward Primer (5′-3′) | CTTTGCCTCACTCAGGATTGG | SEQ ID NO: 89 |
| Ucp1 | Reverse Primer (5′-3′) | CTTTGCCTCACTCAGGATTGG | SEQ ID NO: 90 |
| Ucp2 | Forward Primer (5′-3′) | GTGGTCGGAGATACCAGAGC | SEQ ID NO: 91 |
| Ucp2 | Reverse Primer (5′-3′) | GAGGTTGGCTTTCAGGAGAG | SEQ ID NO: 92 |

Histological Analysis.

Inguinal, epididymal and interscapular adipose tissues were harvested, fixed in 4% (w/v) paraformaldehyde in PBS and embedded in paraffin. Then, 5-μm-thick tissue sections were deparaffinized, rehydrated and used for hematoxylin and eosin (H&E) staining, immunohistochemistry and immunofluorescence. For antigen retrieval, the slides were submerged in 10 mM sodium citrate (pH 6.0) and heated to 95° C. for 20 minutes. Visualization of Ucp1 and Plin1 was performed using a VECTASTAIN ABC Kit (PK-4001, Vector Laboratories, Burlingame, Calif., USA) according to the manufacturer's instructions. Briefly, the slides were incubated with BLOXALL Blocking solution (SP-6000, Vector Laboratories) followed by incubation with 2% normal goat serum for 30 min at room temperature to block non-specific binding. Sections were incubated with primary antibody against Ucp1 (ab10983, Abcam) or Plin1 (ab3526, Abcam) for 1 h at room temperature followed by 30-min incubation with a species-specific, biotinylated secondary antibody. The slides were incubated with Vectastain ABC-AP reagent for 30 min and then incubated with alkaline phosphatase substrate (DAB, SK-4100, Vector Laboratories) for visualization. The stains and antibodies used for the immunofluorescence staining included BODIPY (BODIPY®493/503, Invitrogen), anti-5HT (ab10385, Abcam) and DAPI (D9542, Sigma).

Electron microscopy images of BAT were obtained by transmission electron microscopy (Tecnai Spirit TEM) as previously described³⁵. Briefly, the BAT was first fixed with 2.5% glutaraldehyde, and after fixation, ultra-thin sections were cut, stained with uranyl acetate and lead citrate and then examined under an electron microscope.

Western Blot Analysis.

Whole-cell lysates were extracted by incubating cells in RIPA buffer (25 mM Tris-HCl pH 7.6, 150 mM NaCl, 1% NP-40, 1% sodium deoxycholate, 0.1% SDS) plus protease inhibitors (Roche). Supernatant was collected following a brief centrifuge, and protein concentrations in the supernatant were determined using the BCA Protein Assay Kit (Thermo Scientific, Rockford, Ill., USA). The cell lysates were then mixed with equal volumes of 2× Laemmli buffer (4% SDS, 20% glycerol, 10% 2-mercaptoethanol, 0.01% bromophenol blue, and 120 mM Tris-HCl, pH 6.8) and boiled for 5 min at 95° C. Then, the protein samples were separated by SDS-PAGE and transferred to a PVDF membrane (Millipore). After blocking in a 5% skim milk solution (Sigma), the membranes were incubated with specific primary antibodies, i.e., anti-phospho-Hsl antibody (ser660), an anti-phospho-(Ser/Thr) PKA substrate antibody, and an anti-Act antibody. The membranes were then washed with 1×TBST and incubated with anti-rabbit IgG horseradish peroxidase-linked antibody or anti-mouse IgG antibody. The detection of each protein was performed using Supersignal West Pico Chemiluminescent Substrate (Thermo Scientific), according to the manufacturer's instructions. Signals were captured by a ChemiDoc MP system (Bio-Rad).

Glycerol Release Assay.

Lipolysis was measured as the rate of glycerol release using a free glycerol reagent (Sigma) following the manufacturer's protocol. Briefly, fully differentiated 3T3-L1 adipocytes in a 24-well plate were incubated with 5-HT or DOI in Krebs Ringer phosphate buffer (136 mM NaCl, 4.7 mM KCl, 10 mM NaPO₄, 0.9 mM MgSO₄, and 0.9 mM CaCl₂) containing 4% fatty acid-free bovine serum albumin (Sigma) for 24 h. Isoproterenol was used as a positive control. After incubation, 10 μL of the cell culture supernatant was mixed with 0.8 mL of the free glycerol reagent, and the mixture was then incubated at 37° C. for 5 min. The absorbance of the sample was determined at 540 nm using a spectrophotometer (DU730 Life Science UV/Vis, Beckman Coulter, Indianapolis, Ind., USA). The amount of released glycerol was expressed relative to the cellular protein content.

Data Analysis and Statistics.

All values were expressed as the means and standard error of the means (SEMs). Comparisons between groups were carried out by Student's t test or one-way ANOVA. Normal distribution was tested by f-test. P-values<0.05 were considered statistically significant.

### Results

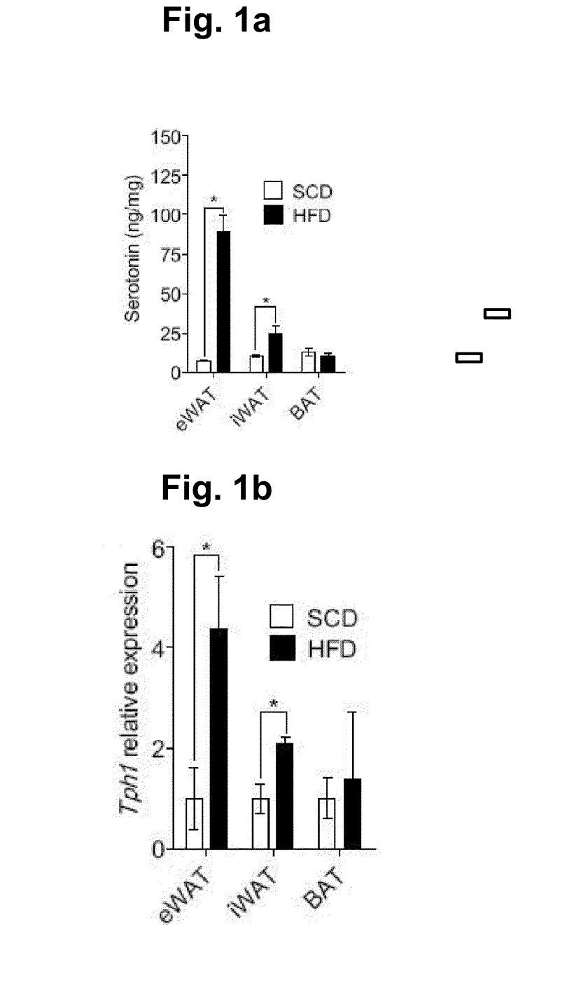

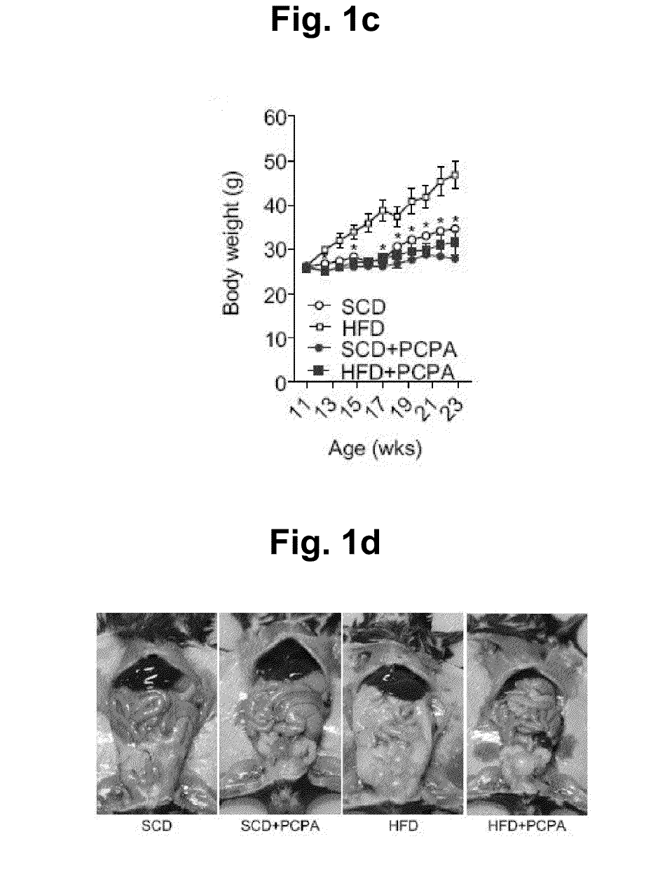

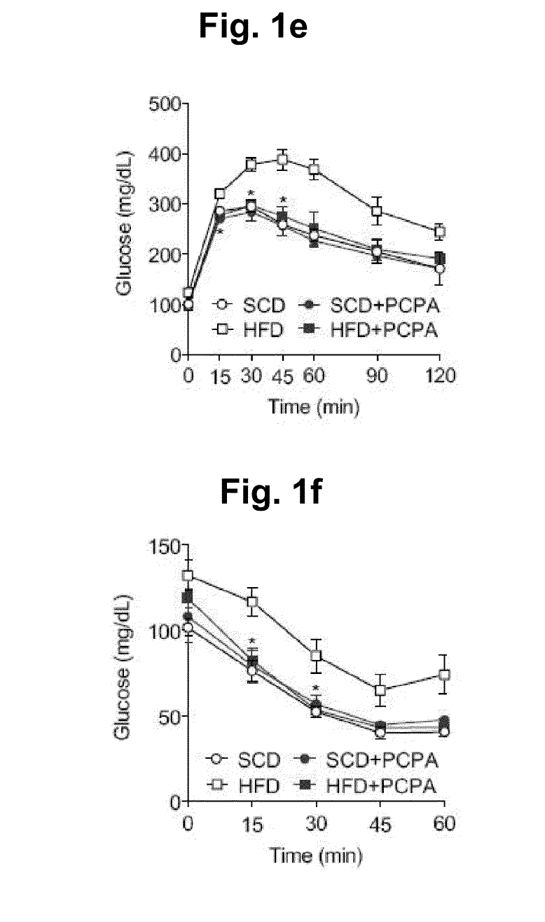

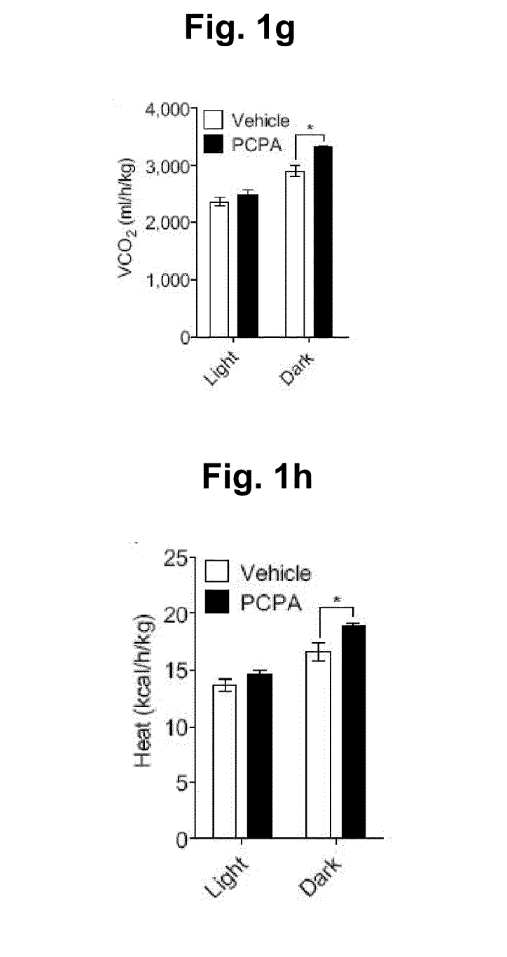

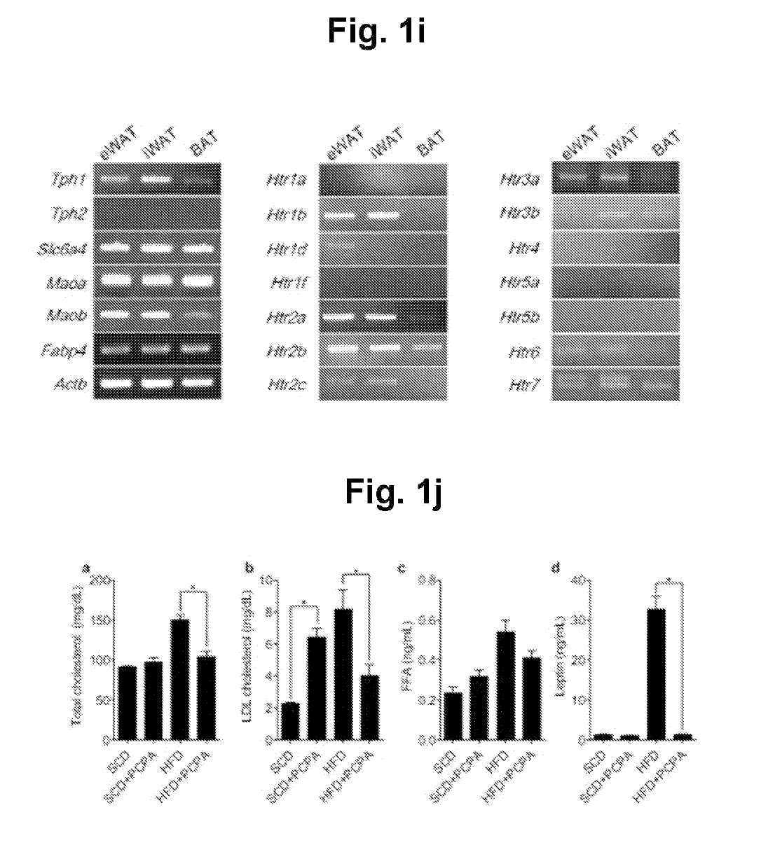

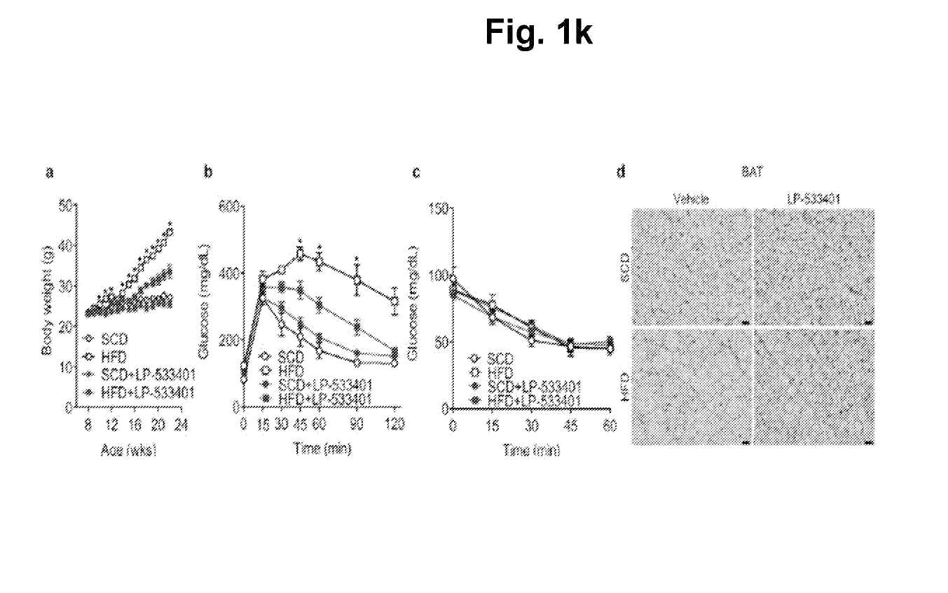

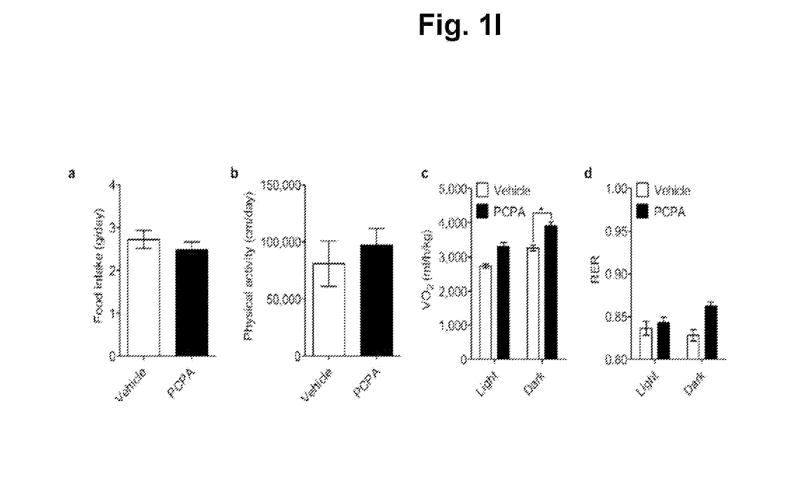

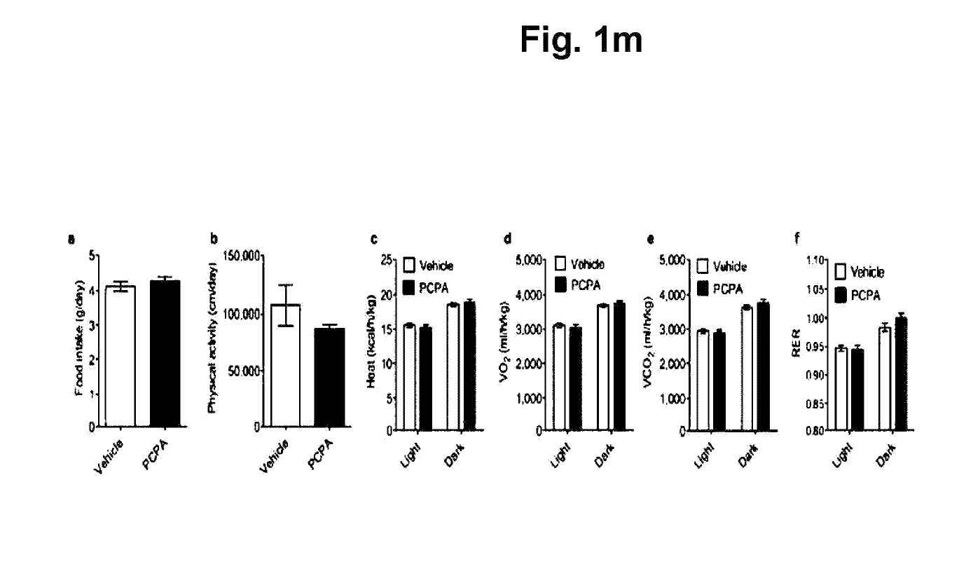

5-HT is known to be present in WAT (white adipose tissue) and BAT (brown adipose tissue) and to promote adipogenesis in 3T3-L1 preadipocytes¹⁵⁻¹⁷. Indeed, 5-HT was detected in adipose tissues, and serotonergic genes, except for Tph2, were expressed in those tissues (FIG. 1a and FIG. 1i). Interestingly, administration of HFD increased tissue 5-HT levels and Tph1 mRNA levels in eWAT (epdidymal white adipose tissue) and iWAT (inguinal white adipose tissue) (FIG. 1a, b), suggesting the potential role of 5-HT in the development of diet-induced obesity. To explore the role of peripheral 5-HT in energy homeostasis, mice were fed a HFD and administered PCPA (p-chlorophenylalanine) by intraperitoneal injection for 10 weeks. As a result of the systemic inhibition of 5-HT synthesis, PCPA-treated mice fed a HFD showed decreased weight gain and reduced eWAT mass compared with WT controls fed a HFD (FIG. 1c, d). Accordingly, PCPA improved glucose tolerance and insulin sensitivity in the HFD-fed mice (FIG. 1e, f). The blood levels of low-density lipoprotein (LDL) cholesterol, free fatty acid (FFA) and leptin were decreased in accordance with the reduction of eWAT in the PCPA-treated mice (FIG. 1j). The anti-obesity effects of PCPA were also observed in mice treated with the peripheral Tph inhibitor LP-533401, which cannot cross the blood-brain barrier¹⁸ (FIG. 1k).

The reduction of eWAT mass observed following PCPA treatment suggests that increased energy expenditure or decreased energy storage may have occurred due to the inhibition of 5-HT synthesis. To address this question, we analyzed the energy consumption rates of the mice using indirect calorimetry after 6 weeks of HFD feeding. PCPA-treated mice showed increased energy expenditure and heat production that could not be attributed to changes in food intake or physical activity (FIG. 1g, 1h and 1l). However, PCPA treatment did not affect the metabolic rates of mice fed a standard chow diet (SCD) (FIG. 1m), suggesting the requirement of metabolic stress for the positive effect of PCPA on energy expenditure. In contrast to our results, intraventricular PCPA injection has been reported to reduce appetite and increase body weight¹⁹. Gut-specific Tph1 KO study has revealed that gut-derived 5-HT is not associated with HFD-induced obesity¹⁴. In this regard, our data suggest that the anti-obesity effects of PCPA resulted from localized 5-HT depletion in adipose tissue, and not from central 5-HT depletion or indirect effects of PCPA.

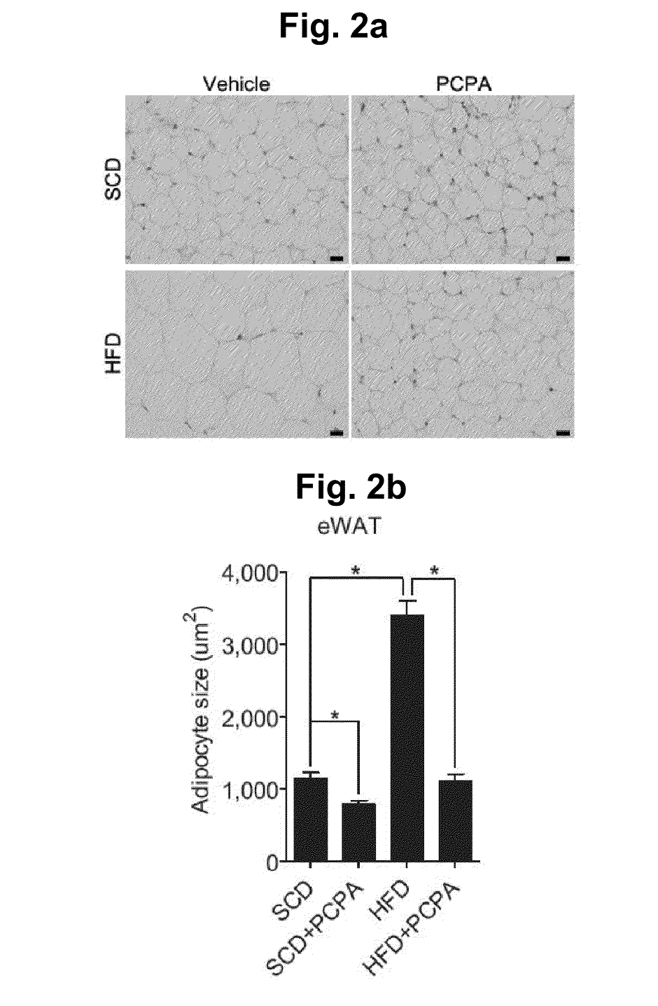

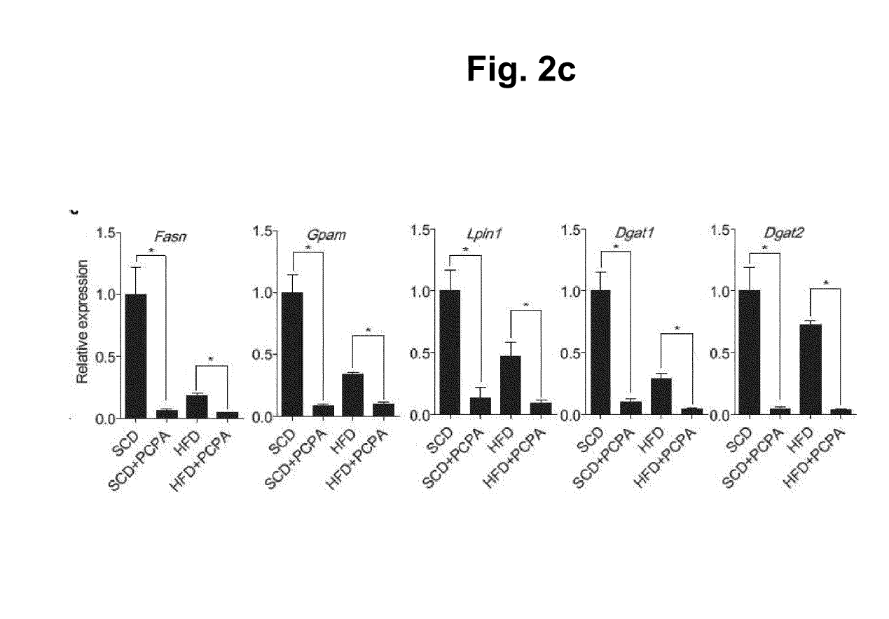

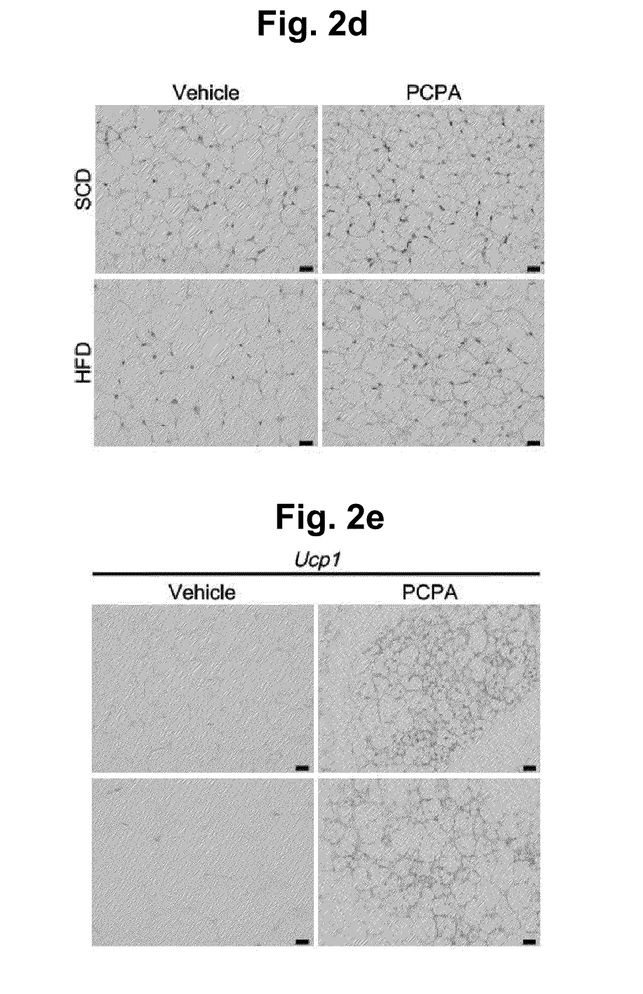

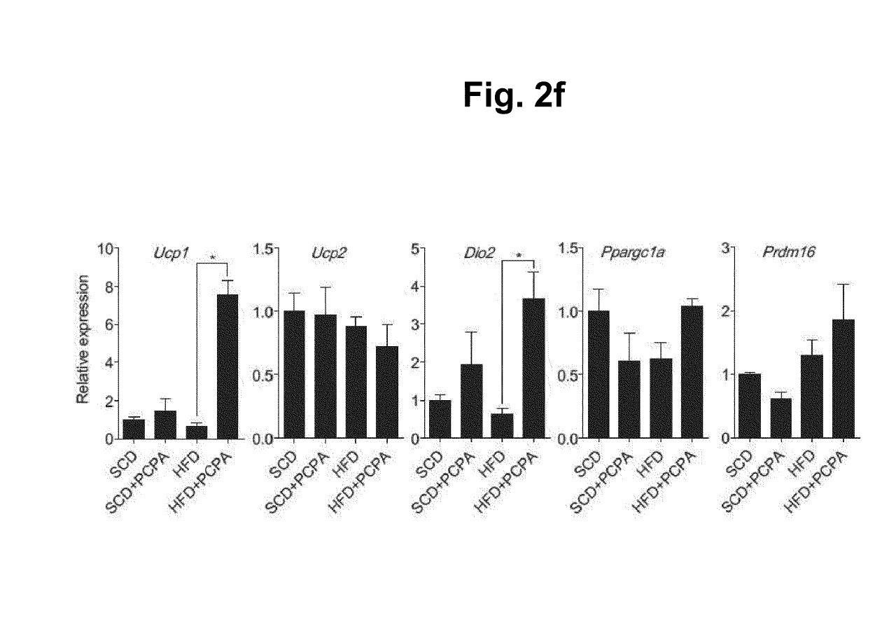

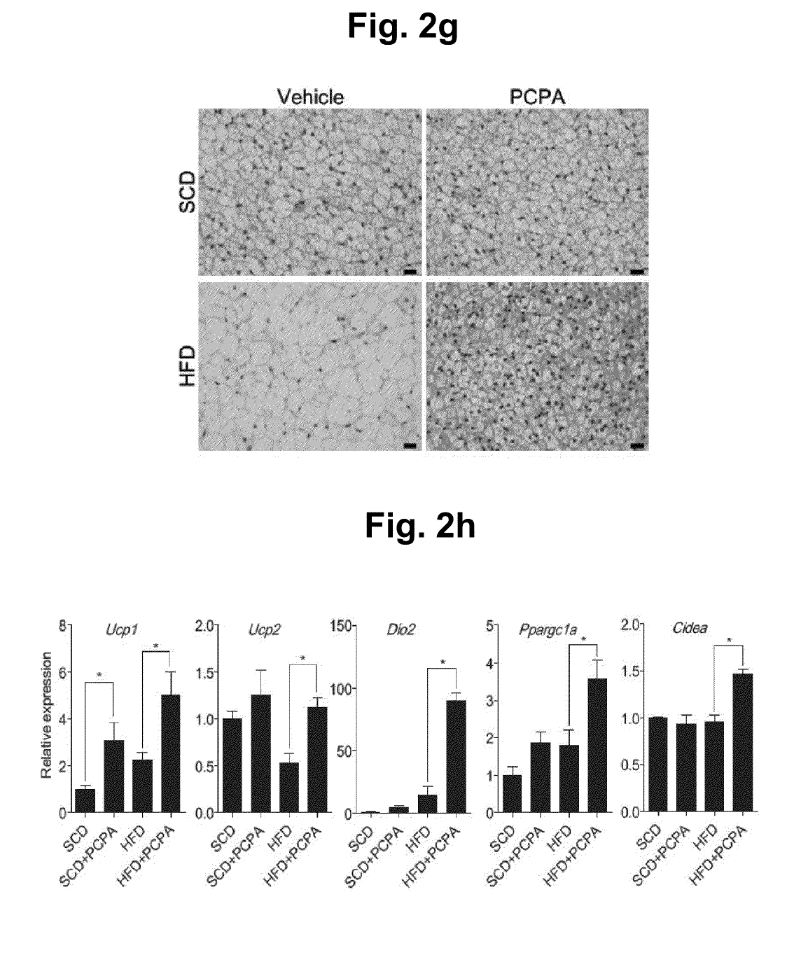

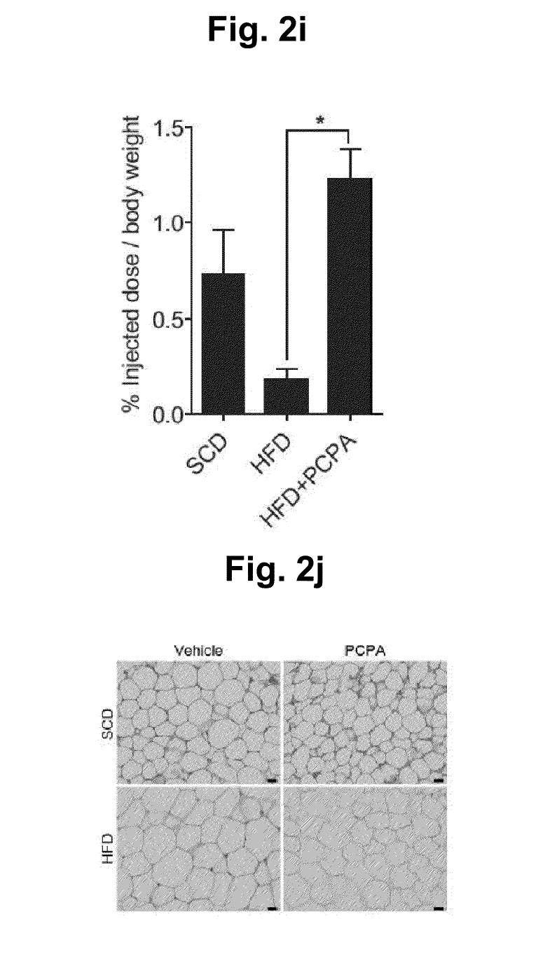

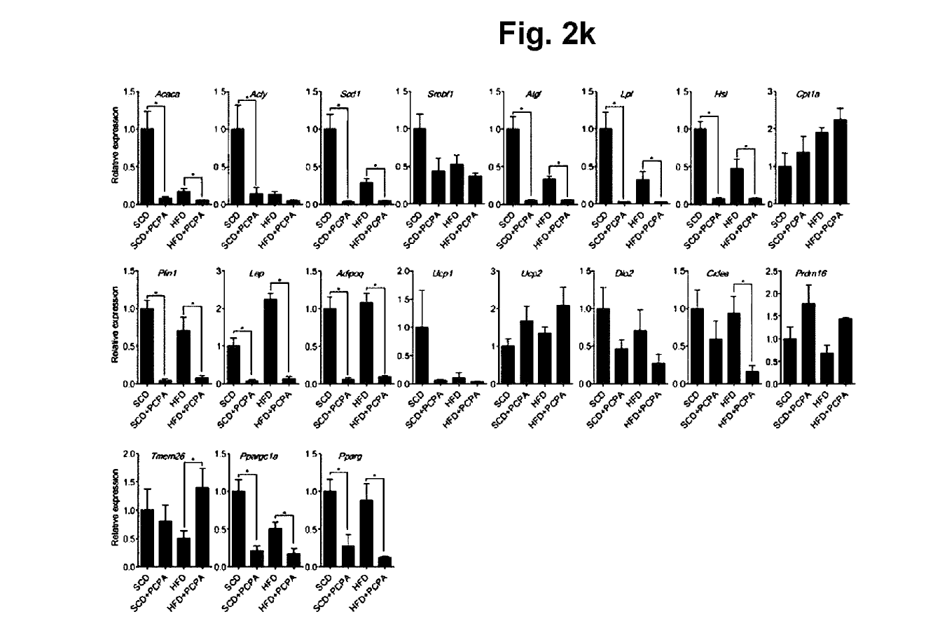

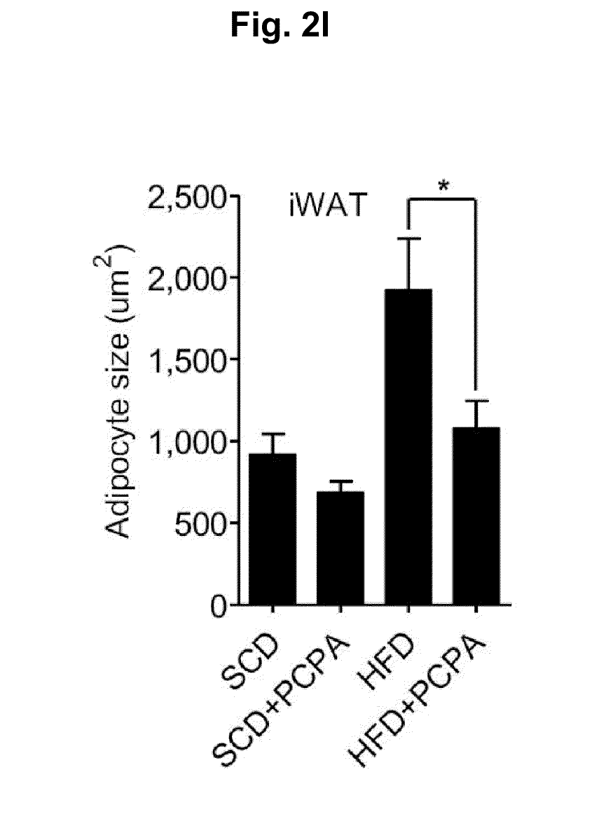

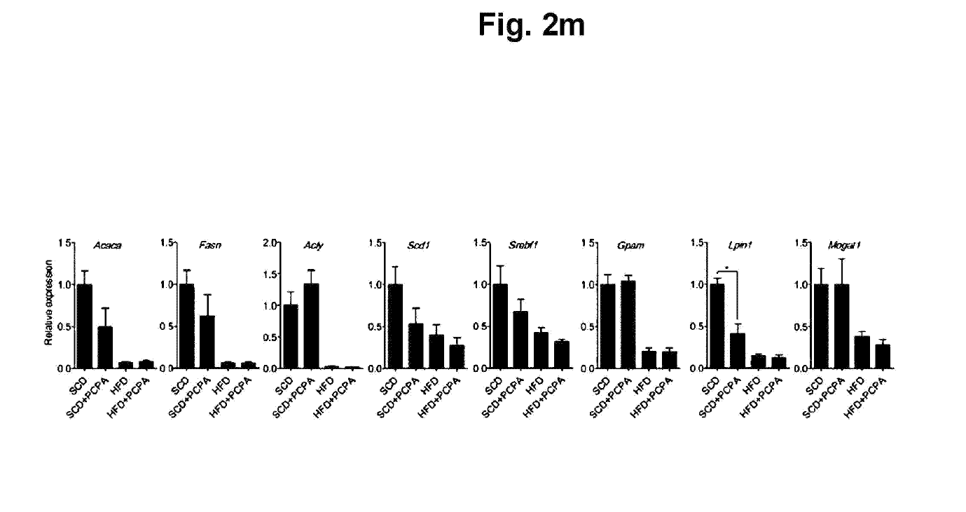

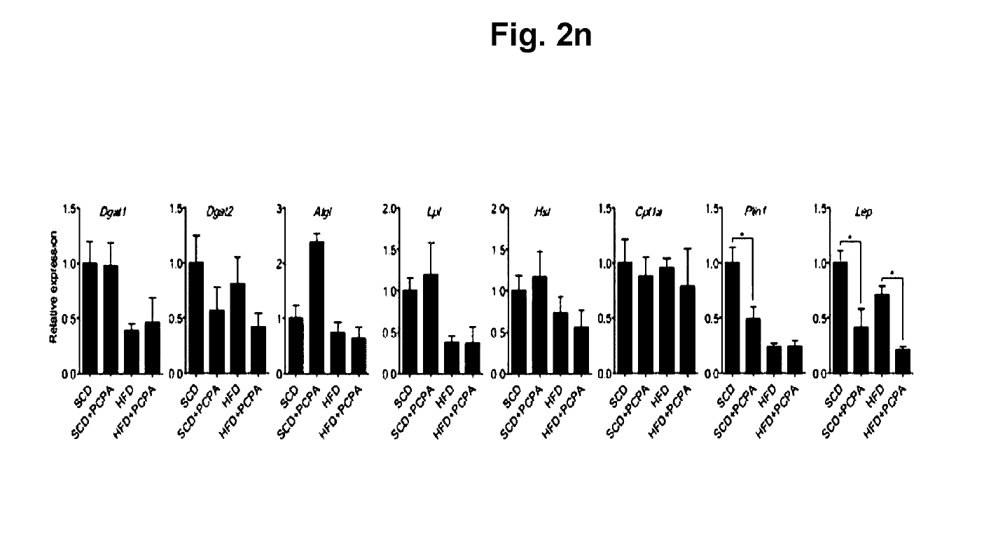

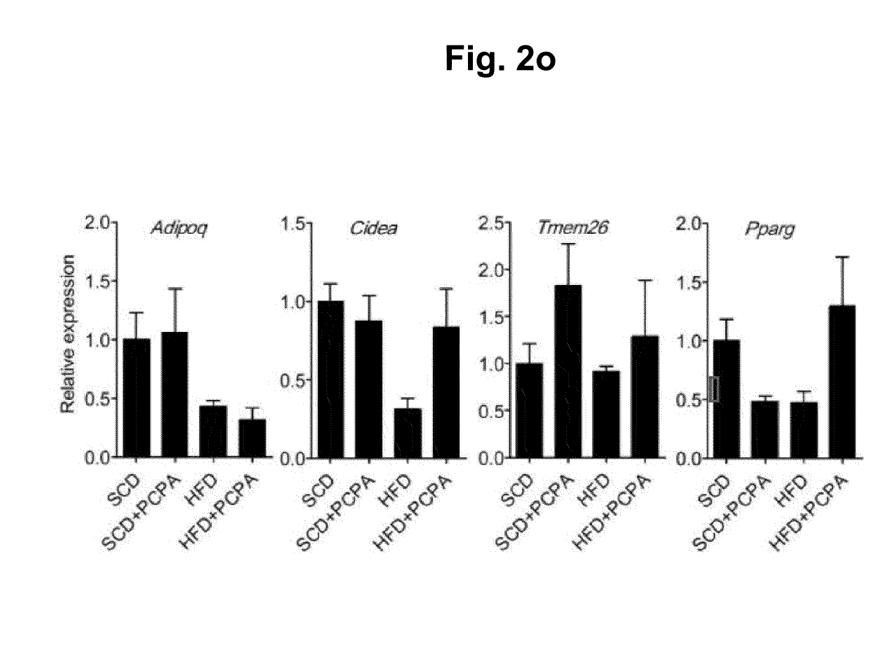

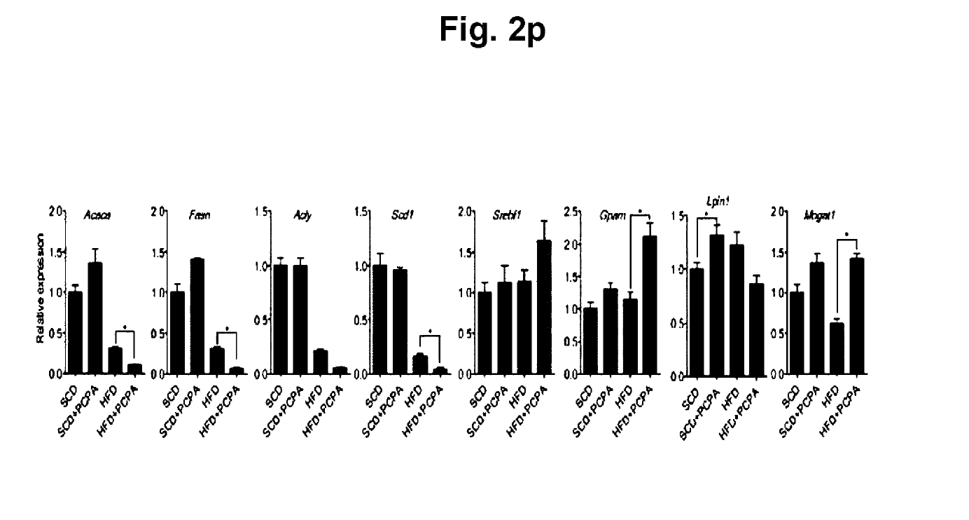

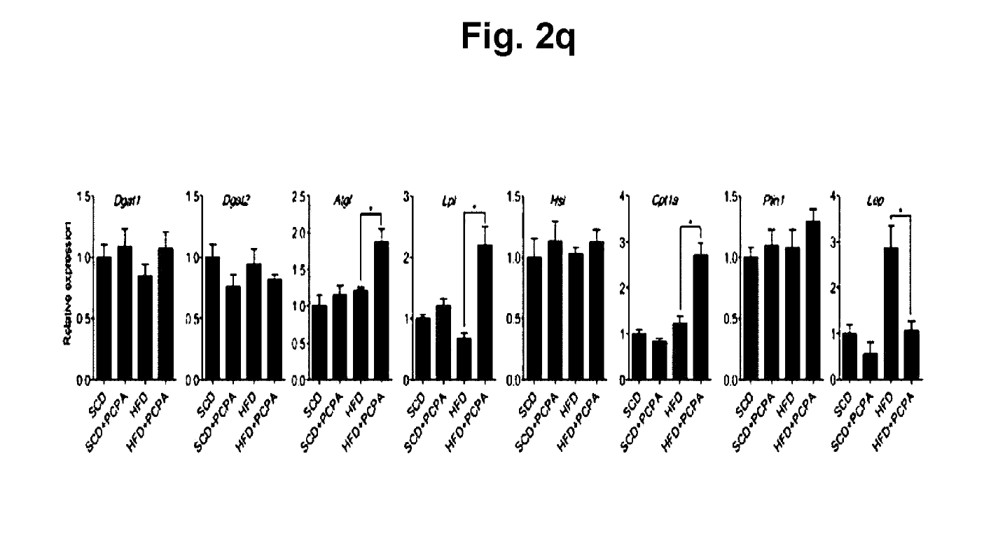

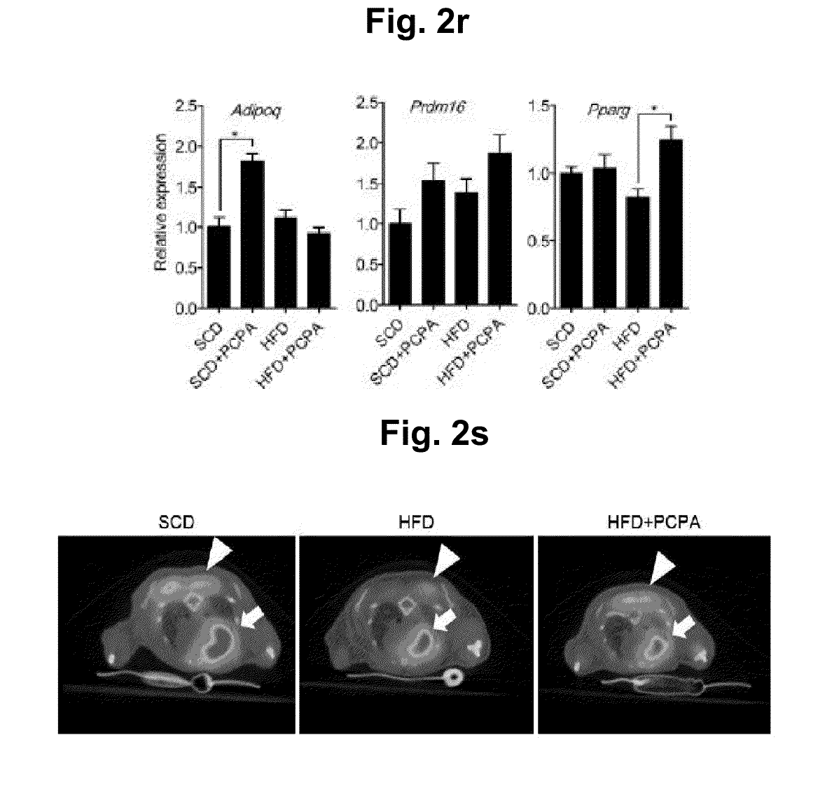

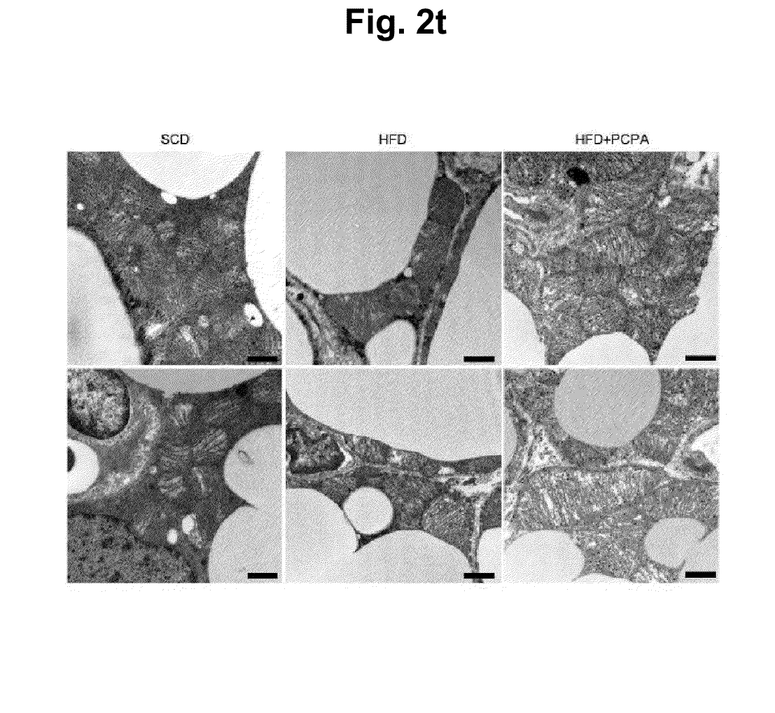

To explore the cell type-specific effects of 5-HT in adipose tissues, we analyzed eWAT, iWAT and BAT. In eWAT, PCPA administration led to decreased adipocyte size but normal cellular structures (FIGS. 2a, 2b and 2j). Real-time RT-PCR analysis of eWAT showed that the expression of most of the genes involved in triglyceride storage was suppressed in PCPA-treated mice compared with control mice, regardless of HFD consumption (FIGS. 2c and 2k). In the iWAT of the PCPA-treated mice, adipocyte size was decreased and brown adipocyte-like multilocular cells that expressed Ucp1 were observed, indicating the browning of the iWAT (FIGS. 2d, e and 2l)²⁰. In agreement with the Ucp1 immunostaining results, Ucp1 and Dio2 mRNA levels were increased in the iWAT following PCPA treatment (FIGS. 2f and 2m-2o). These results suggest that serotonin plays roles in lipogenesis and in the maintenance of WAT.

The ingestion of a HFD leads to dynamic changes in BAT, which involves the enlarged unilocular lipid droplets and the reduction of mitochondrial contents²¹. In this study, the BAT of control mice also showed unilocular lipid droplet formation in brown adipocytes after fed with a HFD, but the BAT from the PCPA-treated mice showed decreased lipid droplet sizes and increased multilocular adipocytes (FIG. 2g). Real time RT-PCR analysis revealed that the PCPA treatment increased thermogenic gene expression in the BAT, and the highest increase was observed in the Dio2 mRNA level (FIGS. 2h and 2p-2r). Because brown adipocytes use glucose as well as lipids to generate heat, the metabolic activity of BAT was measured by assessing ¹⁸fluorodeoxyglucose (¹⁸F-FDG) uptake using positron emission tomography-computed tomography (PET-CT)²². Glucose uptake into BAT is reduced after HFD consumption; however, the inhibition of 5-HT synthesis by PCPA significantly increased glucose uptake into BAT (FIGS. 2i and 2s). In addition, the number and size of the mitochondria and the density of the cristae were increased in BAT of PCPA-treated mice (FIG. 2t).

Since the anti-obesity effects of PCPA were attributed to the potentiation of adaptive thermogenesis in BAT, we attempted to identify the receptors responsible for the activation of BAT. Among the 5-HT receptors (Htr) in BAT (FIG. 1i), we focused on Htr3, which is a heteropentamer of Htr3a and Htr3b that acts as a functional serotonin-gated cation channel^{23,24}. In pancreatic islets, Htr3 activation depolarizes the β-cell membrane, thereby increasing glucose-stimulated insulin secretion²⁵. Because the activation of BAT in response to β3-adrenergic stimulation involves a transient hyperpolarization of membrane potential²⁶, we hypothesized that inhibition of Htr3 could induce membrane hyperpolarization and could have an additive effect on BAT activation. In this regard, we analyzed Htr3a KO mice to assess the role of Htr3 in BAT-adaptive thermogenesis. Htr3a KO mice were resistant to HFD-induced obesity (FIG. 3a), and eWAT mass was reduced (FIG. 3q). Mice were fed an SCD or HFD for 6 weeks from 8 weeks of age. Although the HFD increased visceral fat, HFD-fed Htr3a KO mice had less visceral fat mass than WT littermates. These results were corroborated by the plasma leptin, LDL cholesterol and FFA levels. Htr3a KO mice were fed a SCD or HFD for 6 weeks and blood was obtained from the tail vein. Serum levels of total cholesterol, LDL cholesterol, FFA and leptin were measured by ELISA. Htr3a KO mice fed a HFD showed reduced LDL, leptin but increased FFA level. In contrast to the PCPA-treated mice, the Htr3a KO mice maintained a substantial amount of eWAT mass (FIG. 1j), and no histological differences were observed in the eWAT and iWAT compared to the WT littermates. WAT were isolated from Htr3a KO mice and WT littermates after 6 weeks HFD feeding. Tissue sections were stained with H&E. The average adipocyte size was not significantly decreased in eWAT or iWAT of Htr3a KO mice. HFD-induced enlarged unilocular lipid droplets were observed in the BAT of the WT littermates, but HFD-fed Htr3a KO mice showed decreased adipocyte sizes and increased cell numbers in BAT (FIG. 3b). Furthermore, the number and size of the mitochondria in the BAT of Htr3a KO mice were increased, and the transcription of genes associated with thermogenesis and mitochondrial biogenesis was also increased (FIG. 3c).). Interscapular BAT was isolated from Htr3a KO mice and WT littermates after 6 weeks HFD feeding. Htr3a KO BAT shows increased size and number of mitochondria. Interscapular BAT was isolated from Htr3a KO mice and their WT littermates after 6 weeks HFD feeding. The brown adipocytes mRNA expressions associated with mitochondrial biogenesis were assessed by real time RT-PCR in BAT. Similar to the PCPA-treated mice, Htr3a KO mice exhibited increased energy expenditure and heat production compared to their WT littermates (FIGS. 3d and 3e). The metabolic profiles of Htr3a KO mice and their WT littermates were measured at 14 weeks of age after 6 weeks HFD feeding using the Oxymax system (Columbus instrument). Htr3a KO mice showed increased metabolic rates compared with WT littermates. However, glucose tolerance was not improved in the Htr3a KO mice, despite the improved insulin sensitivity (FIG. 3f, g). Defective insulin secretion in the Htr3a KO mice can explain the discrepancy between glucose tolerance and insulin sensitivity²⁵. These data suggest that the increased mitochondrial biogenesis and energy expenditure observed in BAT resulted from the blocking of Htr3 signaling.

To rule out the effects of Htr3 on the central nervous system, we explored the direct actions of Htr3 on BAT using immortalized brown adipocytes (IBAs). IBA pretreated with the Htr3 antagonist ondansetron showed increased cAMP level and phosphorylation of hormone-sensitive lipase (HSL) and PKA substrate in the presence of β3-adrenergic receptor agonist (FIG. 3h, i). Ondansetron also increased the mRNA expression of thermogenic genes, such as Ucp1 and Ppargc1a, in IBA (FIG. 3j). Conversely, the Htr3 agonist, 1-(m-chlorophenyl)-biguanide (m-CPBG), decreased the phosphorylation of HSL and PKA substrate as well as Ucp1 mRNA in IBA (FIG. 3i, k). We then measured the oxygen consumption rate (OCR) of the IBA. Ondansetron increased the OCR synergistically with the β3-adrenergic receptor agonist (FIG. 3l), which indicated that peripheral 5-HT could directly regulate thermogenesis in BAT through Htr3.

Htr3a KO mice showed reduced eWAT mass, but this reduction was not as severe as that of PCPA-treated mice (FIG. 1j). These results suggest that the effects of Htr3 inhibition are more selective in BAT. To identify an additional mechanism that may explain the severe loss of eWAT following PCPA administration, we performed an in vitro assay using 3T3-L1 preadipocytes, which express Tph1, the expression of which gradually increases during adipocyte differentiation¹⁶. During differentiation, 5-HT was detected after day 4, and interestingly, the expression of Gq-coupled Htr2a gradually increased after day 8 (FIG. 3m). Considering that the preadipocytes were fully differentiated into mature adipocytes after day 8, these results suggest that Htr2a might play a role in lipogenesis in mature adipocytes. Therefore, we investigated whether Htr2a inhibition affects lipogenesis in 3T3-L1 adipocytes. Indeed, the Htr2a agonist 2,5-dimethoxy-4-iodoamphetamine (DOI) increased the mRNA levels of lipogenic genes in mature adipocytes (FIG. 3n). On the other hand, the Htr2a antagonist ketanserin decreased lipogenesis in the mature adipocytes (FIG. 3o). In the glycerol release assay, 5-HT and the Htr2a agonist suppressed lipolysis in mature adipocytes (FIG. 3p), which indicated that 5-HT positively regulates lipogenesis in mature adipocytes through Htr2a.

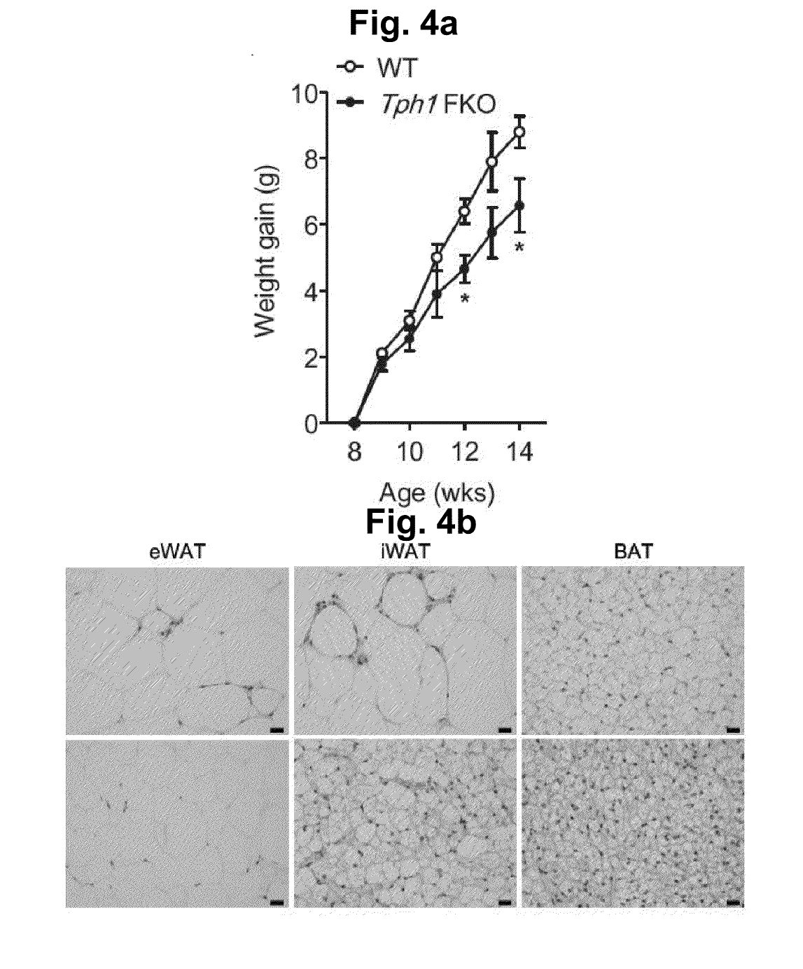

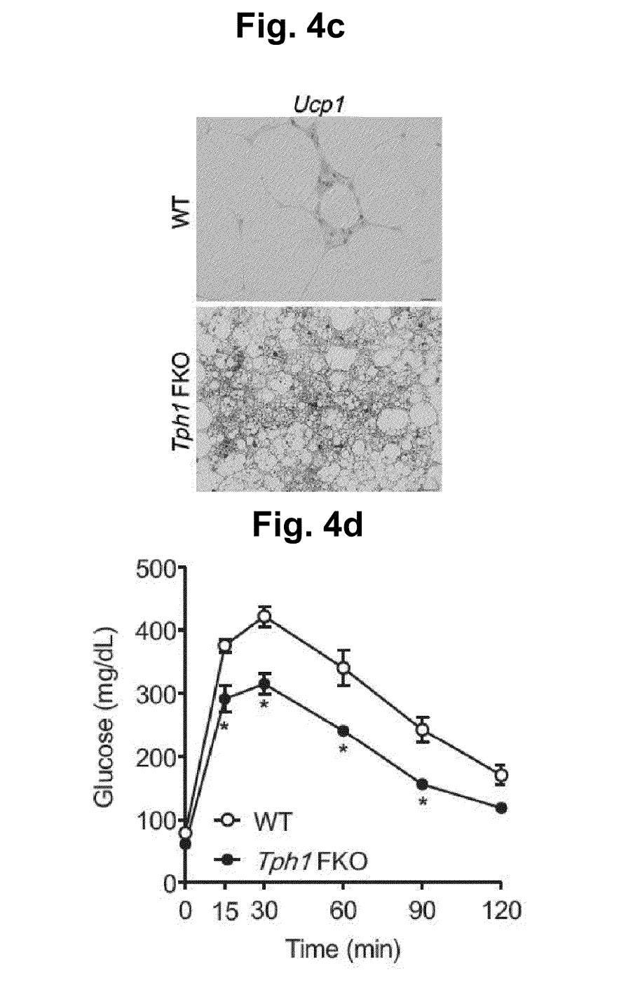

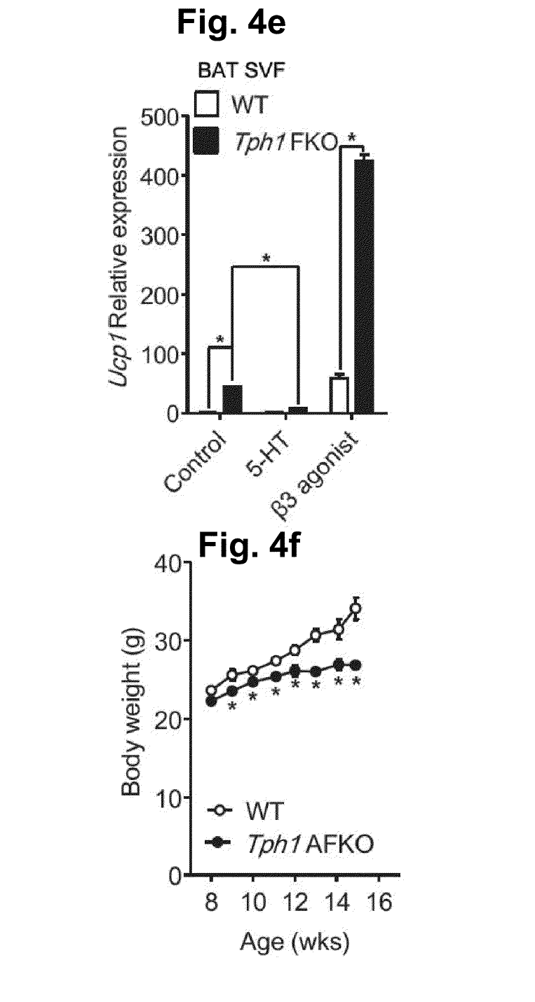

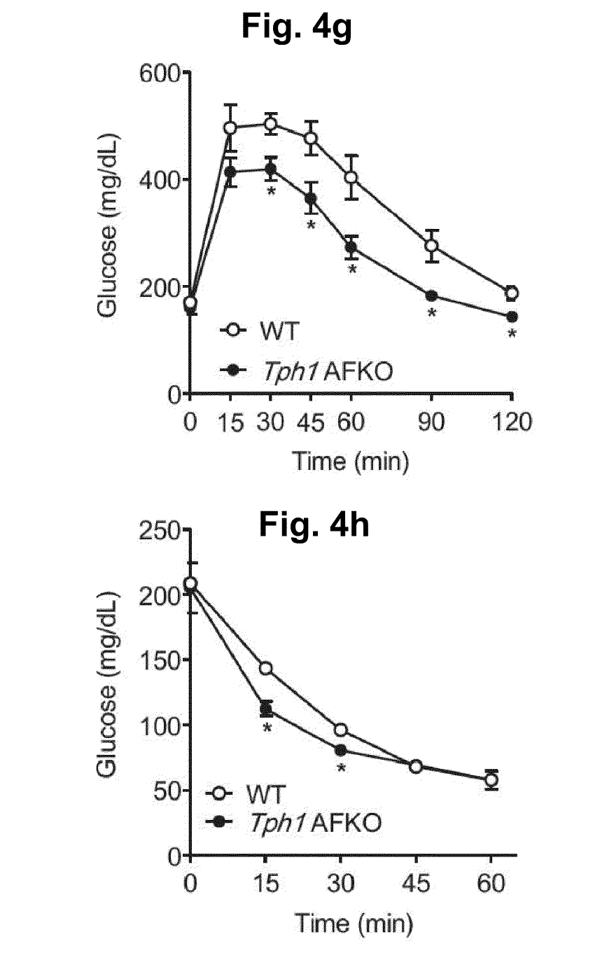

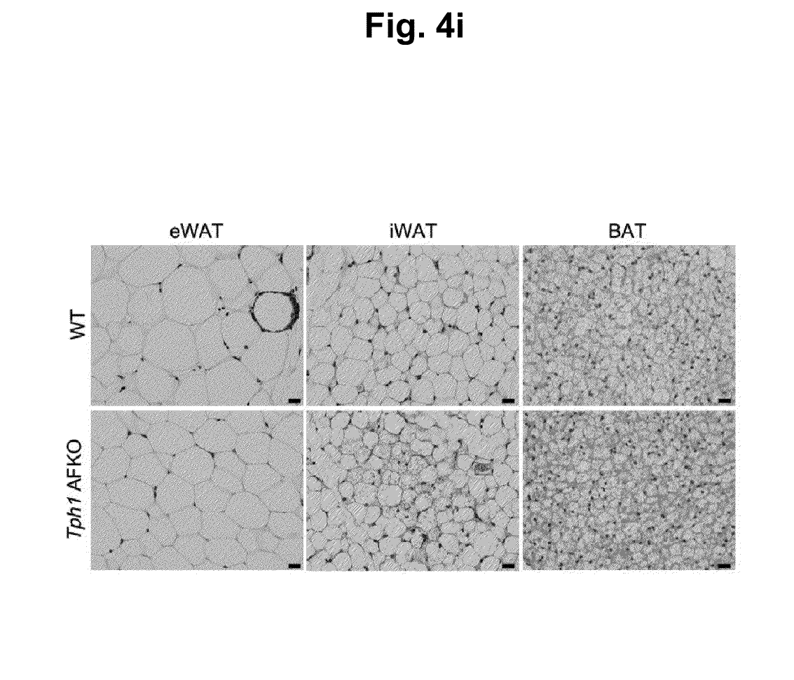

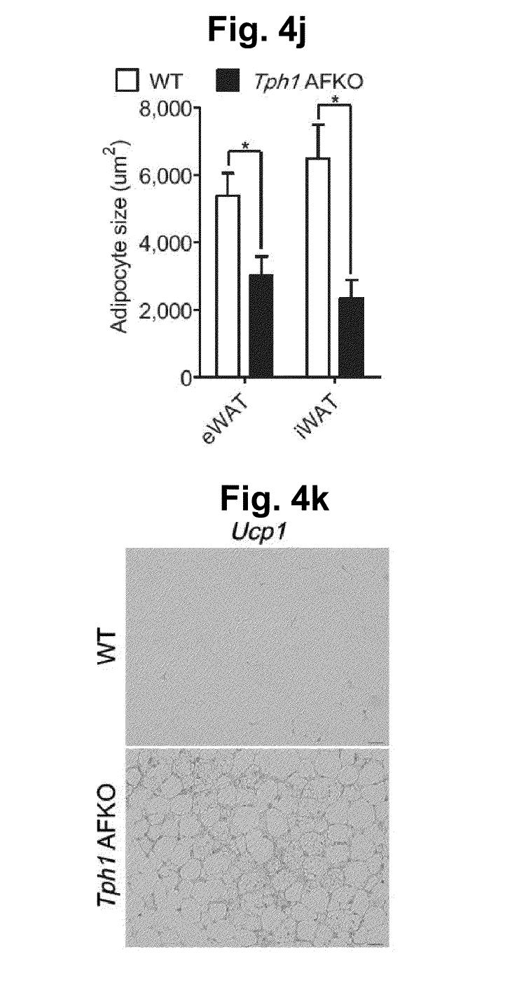

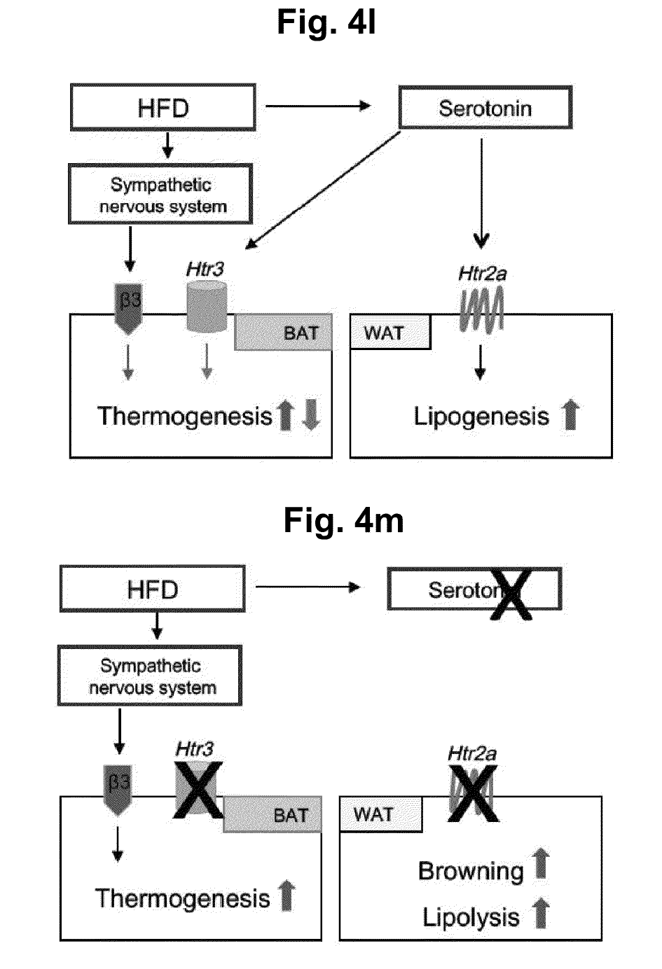

To confirm the cell autonomous function of 5-HT in adipose tissue, we generated adipocyte-specific Tph1 KO (Adipoq-Cre^{+/-}/Tph1^{flox/flox}, Tph1 FKO) mice. Tph1 FKO mice looked grossly normal, and no histological difference was observed in their adipose tissue. However, Tph1 FKO mice showed resistance to HFD-induced obesity and displayed similar histological changes that PCPA-treated mice experienced in both WAT and BAT (FIG. 4a, b). Ucp1 expression was robustly increased in multilocular cells in Tph1-lacking iWAT (FIG. 4c), and glucose tolerance was improved in the Tph1 FKO mice (FIG. 4d). Then, we isolated the stromal vascular fraction (SVF) from Tph1 FKO BAT and tested its potency to differentiate into brown adipocytes. After full differentiation for 8 days in differentiation medium, Ucp1 mRNA expression was upregulated in Tph1 null cells, which was abrogated by 5-HT treatment (FIG. 4e). Furthermore, β3 adrenergic stimulation significantly augmented Ucp1 mRNA expression in the SVF from Tph1 FKO BAT (FIG. 4e). These data suggest the higher potency of brown adipocytes in Tph1 FKO mice²⁹. To test the role of 5-HT in mature adipocytes, we further analyzed the phenotypes of inducible Tph1 FKO (aP2-CreERT2^{+/-}/Tph1^{flox/flox}, Tph1 AFKO) mice. HFD-fed Tph1 AFKO mice also showed reduced weight gain, improved glucose tolerance and insulin sensitivity (FIG. 4f, g, h), and they showed similar histological changes in adipose tissue as PCPA-treated mice (FIGS. 4i, 4j, and 4k). These results demonstrated the cell autonomous role of 5-HT in adipose tissues.

In the present study, we provide a complex model for the regulation of energy metabolism in different adipose tissues (FIG. 4l). In the over-fed state, 5-HT levels increased in the WAT, leading to the augmentation of lipogenesis via Htr2a. The basal 5-HT level also suppressed thermogenesis in the BAT via Htr3a. When 5-HT signaling was blocked, lipolysis increased in the WAT, and thermogenesis increased in both the iWAT and BAT (FIG. 4m). β3 adrenergic signaling stimulated by a HFD coupled with uninhibited thermogenesis induced by the blocking of serotonin-Htr3 signaling resulted in enhanced energy expenditure in both the iWAT and BAT. Thus, the inhibition of 5-HT production in adipose tissues may represent a novel strategy for anti-obesity treatment.

Having described a preferred embodiment of the present invention, it is to be understood that variants and modifications thereof falling within the spirit of the invention may become apparent to those skilled in this art, and the scope of this invention is to be determined by appended claims and their equivalents.

### REFERENCES

1. Tecott, L. H. Serotonin and the orchestration of energy balance. Cell metabolism 6, 352-361 (2007).
2. Berglund, E. D., et al. Serotonin 2C receptors in pro-opiomelanocortin neurons regulate energy and glucose homeostasis. The Journal of clinical investigation 123, 5061 (2013).
3. Nonogaki, K., Strack, A. M., Dallman, M. F. & Tecott, L. H. Leptin-independent hyperphagia and type 2 diabetes in mice with a mutated serotonin 5-HT2C receptor gene. Nat Med 4, 1152-1156 (1998).
4. Sohn, J. W., et al. Serotonin 2C receptor activates a distinct population of arcuate pro-opiomelanocortin neurons via TRPC channels. Neuron 71, 488-497 (2011).
5. Koe, B. K. & Weissman, A. p-Chlorophenylalanine: a specific depletor of brain serotonin. The Journal of pharmacology and experimental therapeutics 154, 499-516 (1966).
6. Rosmond, R., Bouchard, C. & Bjorntorp, P. 5-HT2A Receptor Gene Promoter
Polymorphism in Relation to Abdominal Obesity and Cortisol. Obesity research 10, 585-589 (2002).
7. Chen, C., et al. Genetic Variations in the Serotoninergic System Contribute to Body-Mass Index in Chinese Adolescents. PloS one 8, e58717 (2013).
8. Kwak, S. H., et al. Association of variations in TPH1 and HTR2B with gestational weight gain and measures of obesity. Obesity 20, 233-238 (2011).
9. Murphy, D. L. & Lesch, K. P. Targeting the murine serotonin transporter: insights into human neurobiology. Nature reviews. Neuroscience 9, 85-96 (2008).
10. Savelieva, K. V., et al. Genetic disruption of both tryptophan hydroxylase genes dramatically reduces serotonin and affects behavior in models sensitive to antidepressants. PLoS One 3, e3301 (2008).
11. Yadav, V. K., et al. A serotonin-dependent mechanism explains the leptin regulation of bone mass, appetite, and energy expenditure. Cell 138, 976-989 (2009).
12. Yadav, V. K., et al. Lrp5 controls bone formation by inhibiting serotonin synthesis in the duodenum. Cell 135, 825-837 (2008).
13. Haub, S., et al. Serotonin receptor type 3 antagonists improve obesity-associated fatty liver disease in mice. Journal of Pharmacology and Experimental Therapeutics 339, 790-798 (2011).
14. Sumara, G., Sumara, O., Kim, J. K. & Karsenty, G. Gut-derived serotonin is a multifunctional determinant to fasting adaptation. Cell Metab 16, 588-600 (2012).
15. Omenetti, A., et al. Paracrine modulation of cholangiocyte serotonin synthesis orchestrates biliary remodeling in adults. American Journal of Physiology-Gastrointestinal and Liver Physiology 300, G303-G315 (2011).
16. Kinoshita, M., et al. Regulation of adipocyte differentiation by activation of serotonin (5-HT) receptors 5-HT2AR and 5-HT2CR and involvement of microRNA-448-mediated repression of KLF5. Molecular Endocrinology 24, 1978-1987 (2010).
17. Uchida-Kitajima, S., et al. 5-Hydroxytryptamine 2A receptor signaling cascade modulates adiponectin and plasminogen activator inhibitor 1 expression in adipose tissue. FEBS letters 582, 3037-3044 (2008).
18. Liu, Q., et al. Discovery and characterization of novel tryptophan hydroxylase inhibitors that selectively inhibit serotonin synthesis in the gastrointestinal tract. Journal of Pharmacology and Experimental Therapeutics 325, 47-55 (2008).
19. Breisch, S. T., Zemlan, F. P. & Hoebel, B. G. Hyperphagia and obesity following serotonin depletion by intraventricular p-chlorophenylalanine. Science 192, 382-385 (1976).
20. Wu, J., et al. Beige adipocytes are a distinct type of thermogenic fat cell in mouse and human. Cell 150, 366-376 (2012).
21. Shimizu, I., et al. Vascular rarefaction mediates whitening of brown fat in obesity. The Journal of clinical investigation 124, 2099-2112 (2014).
22. Nicholls, D. G. & Locke, R. M. Thermogenic mechanisms in brown fat. Physiological reviews 64, 1-64 (1984).
23. Maricq, A. V., Peterson, A. S., Brake, A. J., Myers, R. M. & Julius, D. Primary structure and functional expression of the 5HT3 receptor, a serotonin-gated ion channel. Science 254, 432-437 (1991).
24. Derkach, V., Surprenant, A. & North, R. A. 5-HT3 receptors are membrane ion channels. Nature 339, 706-709 (1989).
25. Ohara-Imaizumi, M., et al. Serotonin regulates glucose-stimulated insulin secretion from pancreatic β cells during pregnancy. Proceedings of the National Academy of Sciences 110, 19420-19425 (2013).
26. Girardier, L. & Schneider-Picard, G. Alpha and beta-adrenergic mediation of membrane potential changes and metabolism in rat brown adipose tissue. The Journal of physiology 335, 629-641 (1983).
27. Rosmond, R. Association studies of genetic polymorphisms in central obesity: a critical review. International journal of obesity 27, 1141-1151 (2003).
28. Li, P., et al. Genetic association analysis of 30 genes related to obesity in a European American population. International Journal of Obesity (2013).
29. Seale, P., et al. Prdm16 determines the thermogenic program of subcutaneous white adipose tissue in mice. The Journal of clinical investigation 121, 96-105 (2011).
30. Eguchi, J., et al. Transcriptional control of adipose lipid handling by IRF4. Cell metabolism 13, 249-259 (2011).
31. Imai, T., Jiang, M., Chambon, P. & Metzger, D. Impaired adipogenesis and lipolysis in the mouse upon selective ablation of the retinoid X receptor α mediated by a tamoxifen-inducible chimeric Cre recombinase (Cre-ERT2) in adipocytes. Proceedings of the National Academy of Sciences 98, 224-228 (2001).
32. Uldry, M., et al. Complementary action of the PGC-1 coactivators in mitochondrial biogenesis and brown fat differentiation. Cell metabolism 3, 333-341 (2006).
33. Bernal-Mizrachi, C., et al. Respiratory uncoupling lowers blood pressure through a leptin-dependent mechanism in genetically obese mice. Arteriosclerosis, thrombosis, and vascular biology 22, 961-968 (2002).
34. Wang, X., Minze, L. & Shi, Z. Functional imaging of brown fat in mice with 18F-FDG micro-PET/CT. Journal of visualized experiments: JoVE (2011).
35. Ozaki, K., Sano, T., Tsuji, N., Matsuura, T. & Narama, I. Carnitine is necessary to maintain the phenotype and function of brown adipose tissue. Laboratory Investigation 91, 704-710 (2011).

What is claimed is:

1. A method for preventing or treating a metabolic disease, which comprises inhibiting HTR2A (5-hydroxytryptamine 2A receptor) or HTR3 (5-hydroxytryptamine 3 receptor) in a subject in need thereof.

2. The method according to claim 1, wherein the inhibition of HTR2A or HTR3 is performed by suppressing the expression of HTR2A or HTR3A.

3. The method according to claim 1, wherein the inhibition of HTR2A or HTR3 is performed by administering to the subject an antagonist against HTR2A or HTR3A.

4. The method according to claim 4, wherein the antagonist against HTR2A is ketanserin, ritanserin, nefazodone, clozapine, olanzapine, quetiapine, risperidone, asenapine, volinanserin, or AMDA.

5. The method according to claim 4, wherein the antagonist against HTR3A is ondansetron, granisetron, tropisetron, dolasetron, palonosetron, ramosetron, alosetron, batanopride, renzapride or zacopride.

6. The method according to claim 1, wherein the inhibition of HTR2A or HTR3 is performed together with activating β3-adrenergic receptor.

7. The method according to claim 6, wherein the activation of β3-adrenergic receptor is performed by administering to the subject an agonist for β3-adrenergic receptor.

8. The method according to claim 7, wherein the agonist for β3-adrenergic receptor is 5-[(2R)-2-[[(2R)-2-(3-Chlorophenyl)-2-hydroxyethyl]amino]propyl]-1,3-benzodioxole-2,2-dicarboxylic acid; amibegron; mirabegron; solabegron; N-[4-[2-[[(2S)-2-hydroxy-3-(4-hydroxyphenoxy)propyl]amino]ethyl]phenyl]-4-iodobenzenesulfonamide; or (R)—N-[4-[2-[[2-hydroxy-2-(3-pyridinyl)ethyl]amino]ethyl]-phenyl]-4-[4-[4-(trifluoromethyl)phenyl]thiazol-2-yl]-benzenesulfonamide, dihydrochloride].

9. The method according to claim 1, wherein HTR2A is present in a white adipose tissue (WAT) and HTR3 is present in a brown adipose tissue (BAT).

10. The method according to claim 1, wherein the metabolic disease is obesity, diabetes, insulin resistance, hyperlipidemia or hypercholesterolemia.

11. A method for screening a therapeutic agent for treating a metabolic disease, comprising:
(a) contacting a test substance of interest for analysis to HTR2A (5-hydroxytryptamine 2a receptor) or HTR3 (5-hydroxytryptamine 3 receptor); and
(b) analyzing whether the test substance inhibits HTR2A or HTR3;
wherein where the test substance inhibits HTR2A or HTR3, it is determined as the therapeutic agent for treating the metabolic disease.

12. The method according to claim 11, wherein the inhibition of HTR2A or HTR3 is suppression of the expression of HTR2A or HTR3A.

13. The method according to claim 11, wherein the inhibition of HTR2A or HTR3 is suppression of function of HTR2A or HTR3A.

14. The method according to claim 11, wherein HTR2A is present in a white adipose cell and HTR3 is present in a brown adipose cell.

15. The method according to claim 14, wherein the contacting of the test substance to HTR3 in the BAT is performed together with activating β3-adrenergic receptor.

16. The method according to claim 11, wherein the metabolic disease is obesity, diabetes, insulin resistance, hyperlipidemia or hypercholesterolemia.


SEQUENCE LISTING (92 sequences)

SEQ ID NO: 1 - DNA, 21 bp, Artificial Sequence
  Forward primer for Acaca
cagtaacctggtgaagctgga

SEQ ID NO: 2 - DNA, 21 bp, Artificial Sequence
  Reverse primer for Acaca
gccagacatgctggatctcat

SEQ ID NO: 3 - DNA, 20 bp, Artificial Sequence
  Forward primer for Acly
ccctcttcagccgacatacc

SEQ ID NO: 4 - DNA, 20 bp, Artificial Sequence
  Reverse primer for Acly
ctgcttgtgatccccagtga

SEQ ID NO: 5 - DNA, 20 bp, Artificial Sequence
  Forward primer for Actb
cagcttctttgcagctcctt

SEQ ID NO: 6 - DNA, 20 bp, Artificial Sequence
  Reverse primer for Actb
cttctccatgtcgtcccagt

SEQ ID NO: 7 - DNA, 20 bp, Artificial Sequence
  Forward primer for Adipoq
ctccacccaagggaacttgt

SEQ ID NO: 8 - DNA, 20 bp, Artificial Sequence
  Reverse primer for Adipoq
ggaccaagaagacctgcatc

SEQ ID NO: 9 - DNA, 21 bp, Artificial Sequence
  Forward primer for Cidea
gccgtgttaaggaatctgctg

SEQ ID NO: 10 - DNA, 21 bp, Artificial Sequence
  Reverse primer for Cidea
tgctcttctgtatcgcccagt

SEQ ID NO: 11 - DNA, 20 bp, Artificial Sequence
  Forward primer for Cox8b
gaaccatgaagccaacgact

SEQ ID NO: 12 - DNA, 20 bp, Artificial Sequence
  Reverse primer for Cox8b
gcgaagttcacagtggttcc

SEQ ID NO: 13 - DNA, 21 bp, Artificial Sequence
  Forward primer for Cptla
agctcgcacattacaaggaca

SEQ ID NO: 14 - DNA, 20 bp, Artificial Sequence
  Reverse primer for Cptla
ccagcacaaagttgcaggac

SEQ ID NO: 15 - DNA, 22 bp, Artificial Sequence
  Forward primer for Cycs
gcaagcataagactggaccaaa

SEQ ID NO: 16 - DNA, 25 bp, Artificial Sequence
  Reverse primer for Cycs
ttgttggcatctgtgtaagagaatc

SEQ ID NO: 17 - DNA, 20 bp, Artificial Sequence
  Forward primer for Dgat1
ggatctgaggtgccatcgtc

SEQ ID NO: 18 - DNA, 20 bp, Artificial Sequence
  Reverse primer for Dgat1
atcagcatcaccacacacca

SEQ ID NO: 19 - DNA, 20 bp, Artificial Sequence
  Forward primer for Dgat2
catcatcgtggtgggaggtg

SEQ ID NO: 20 - DNA, 21 bp, Artificial Sequence
  Reverse primer for Dgat2
tgggaaccagatcagctccat

SEQ ID NO: 21 - DNA, 20 bp, Artificial Sequence
  Forward primer for Dio2
ttggggtagggaatgttggc

SEQ ID NO: 22 - DNA, 20 bp, Artificial Sequence
  Reverse primer for Dio2
tccgtttcctctttccggtg

SEQ ID NO: 23 - DNA, 20 bp, Artificial Sequence
  Forward primer for Fabp4
aacaccgagatttccttcaa

SEQ ID NO: 24 - DNA, 20 bp, Artificial Sequence
  Reverse primer for Fabp4
tcacgcctttcataacacat

SEQ ID NO: 25 - DNA, 20 bp, Artificial Sequence
  Forward primer for Fasn
aagcggtctggaaagctgaa

SEQ ID NO: 26 - DNA, 20 bp, Artificial Sequence
  Reverse primer for Fasn
aggctgggttgatacctcca

SEQ ID NO: 27 - DNA, 20 bp, Artificial Sequence
  Forward primer for Gpam
ccacagagctgggaaaggtt

SEQ ID NO: 28 - DNA, 20 bp, Artificial Sequence
  Reverse primer for Gpam
gtgccttgtgtgcgtttcat

SEQ ID NO: 29 - DNA, 20 bp, Artificial Sequence
  Forward primer for Hsl
aacgagacaggcctcagtgt

SEQ ID NO: 30 - DNA, 20 bp, Artificial Sequence
  Reverse primer for Hsl
gaatcggccaccggtaaaga

SEQ ID NO: 31 - DNA, 23 bp, Artificial Sequence
  Forward primer for Htr1a
tcagctaccaagtgatcacctct

SEQ ID NO: 32 - DNA, 20 bp, Artificial Sequence
  Reverse primer for Htr1a
gtccacttgttgagcacctg

SEQ ID NO: 33 - DNA, 21 bp, Artificial Sequence
  Forward primer for Htr1b
tgctcctcatcgccctctatg

SEQ ID NO: 34 - DNA, 23 bp, Artificial Sequence
  Reverse primer for Htr1b
ctagcggccatgagtttcttctt

SEQ ID NO: 35 - DNA, 21 bp, Artificial Sequence
  Forward primer for Htr1d
cctccaacagatccctgaatg

SEQ ID NO: 36 - DNA, 23 bp, Artificial Sequence
  Reverse primer for Htr1d
cagagcaatgacacagagatgca

SEQ ID NO: 37 - DNA, 23 bp, Artificial Sequence
  Forward primer for Htr1f
tgtgagagagagctggattatgg

SEQ ID NO: 38 - DNA, 22 bp, Artificial Sequence
  Reverse primer for Htr1f
tagttccttggtgcctccagaa

SEQ ID NO: 39 - DNA, 23 bp, Artificial Sequence
  Forward primer for Htr2a
agctgcagaatgccaccaactat

SEQ ID NO: 40 - DNA, 23 bp, Artificial Sequence
  Reverse primer for Htr2a
gggattggcatggatatacctac

SEQ ID NO: 41 - DNA, 21 bp, Artificial Sequence
  Forward primer for Htr2b
aaataagccacctcaacgcct

SEQ ID NO: 42 - DNA, 23 bp, Artificial Sequence
  Reverse primer for Htr2b
tcccgaaatgtcttattgaagag

SEQ ID NO: 43 - DNA, 23 bp, Artificial Sequence
  Forward primer for Htr2c
ttcttaatgtccctagccattgc

SEQ ID NO: 44 - DNA, 23 bp, Artificial Sequence
  Reverse primer for Htr2c
gcaatcttcatgatggccttagt

SEQ ID NO: 45 - DNA, 22 bp, Artificial Sequence
  Forward primer for Htr3a
aaatcagggcgagtgggagctg

SEQ ID NO: 46 - DNA, 22 bp, Artificial Sequence
  Reverse primer for Htr3a
gacacgatgatgaggaagactg

SEQ ID NO: 47 - DNA, 20 bp, Artificial Sequence
  Forward primer for Htr3b
cgtgtggtaccgagaggttt

SEQ ID NO: 48 - DNA, 20 bp, Artificial Sequence
  Reverse primer for Htr3b
ggatgggcttgtggtttcta

SEQ ID NO: 49 - DNA, 25 bp, Artificial Sequence
  Forward primer for Htr4
atggacaaacttgatgctaatgtga

SEQ ID NO: 50 - DNA, 21 bp, Artificial Sequence
  Reverse primer for Htr4
tcaccagcaccgaaaccagca

SEQ ID NO: 51 - DNA, 20 bp, Artificial Sequence
  Forward primer for Htr5a
gattgacttcagtgggctcg

SEQ ID NO: 52 - DNA, 20 bp, Artificial Sequence
  Reverse primer for Htr5a
aaagtcaggactagcactcg

SEQ ID NO: 53 - DNA, 20 bp, Artificial Sequence
  Forward primer for Htr7
ctcggtgtgctttgtcaaga

SEQ ID NO: 54 - DNA, 20 bp, Artificial Sequence
  Reverse primer for Htr7
ttggccatacatttcccatt

SEQ ID NO: 55 - DNA, 19 bp, Artificial Sequence
  Forward primer for Lep
acacacgcagtcggtatcc

SEQ ID NO: 56 - DNA, 20 bp, Artificial Sequence
  Reverse primer for Lep
gcagcacattttgggaaggc

SEQ ID NO: 57 - DNA, 20 bp, Artificial Sequence
  Forward primer for Lpin1
catacaaaggcagccacacg

SEQ ID NO: 58 - DNA, 20 bp, Artificial Sequence
  Reverse primer for Lpin1
catacaaaggcagccacacg

SEQ ID NO: 59 - DNA, 20 bp, Artificial Sequence
  Forward primer for Maoa
gcggtacaagggtctgttcc

SEQ ID NO: 60 - DNA, 20 bp, Artificial Sequence
  Reverse primer for Maoa
cagccaatcctgagatgccg

SEQ ID NO: 61 - DNA, 20 bp, Artificial Sequence
  Forward primer for Maob
gggcggcatctcaggtatgg

SEQ ID NO: 62 - DNA, 20 bp, Artificial Sequence
  Reverse primer for Maob
aagtcctgcctcctacacgg

SEQ ID NO: 63 - DNA, 20 bp, Artificial Sequence
  Forward primer for Me1
gacccgcatctcaacaagga

SEQ ID NO: 64 - DNA, 22 bp, Artificial Sequence
  Reverse primer for Me1
caggagatacctgtcgaagtca

SEQ ID NO: 65 - DNA, 25 bp, Artificial Sequence
  Forward primer for Nrf1
cagcaaccctgatggcaccgtgtcg

SEQ ID NO: 66 - DNA, 25 bp, Artificial Sequence
  Reverse primer for Nrf1
ggcctctgatgcttgcgtcgtctgg

SEQ ID NO: 67 - DNA, 21 bp, Artificial Sequence
  Forward primer for Plin1
ggtgttacagcgtggagagta

SEQ ID NO: 68 - DNA, 20 bp, Artificial Sequence
  Reverse primer for Plin1
tctggaagcactcacaggtc

SEQ ID NO: 69 - DNA, 21 bp, Artificial Sequence
  Forward primer for Pparg
ggtgtgatcttaactgccgga

SEQ ID NO: 70 - DNA, 20 bp, Artificial Sequence
  Reverse primer for Pparg
gcccaaacctgatggcattg

SEQ ID NO: 71 - DNA, 20 bp, Artificial Sequence
  Forward primer for Ppargc1a
gcccaggtacgacagctatg

SEQ ID NO: 72 - DNA, 20 bp, Artificial Sequence
  Reverse primer for Ppargc1a
acggcgctcttcaattgctt

SEQ ID NO: 73 - DNA, 20 bp, Artificial Sequence
  Forward primer for Prdm16
agccctcgcccacaacttgc

SEQ ID NO: 74 - DNA, 20 bp, Artificial Sequence
  Reverse primer for Prdm16
tgacccccggcttccgttca

SEQ ID NO: 75 - DNA, 20 bp, Artificial Sequence
  Forward primer for Scd1
agagtcaggagggcaggttt

SEQ ID NO: 76 - DNA, 22 bp, Artificial Sequence
  Reverse primer for Scd1
gaactggagatctcttggagca

SEQ ID NO: 77 - DNA, 19 bp, Artificial Sequence
  Forward primer for Slc6a4
cgcagttcccagtacaagc

SEQ ID NO: 78 - DNA, 19 bp, Artificial Sequence
  Reverse primer for Slc6a4
cgtgaaggaggagatgagg

SEQ ID NO: 79 - DNA, 18 bp, Artificial Sequence
  Forward primer for Srebf1
gtgggcctagtccgaagc

SEQ ID NO: 80 - DNA, 20 bp, Artificial Sequence
  Reverse primer for Srebf1
ctggagcatgtcttcgatgt

SEQ ID NO: 81 - DNA, 20 bp, Artificial Sequence
  Forward primer for Tfam
agttcccacgctggtagtgt

SEQ ID NO: 82 - DNA, 16 bp, Artificial Sequence
  Reverse primer for Tfam
gcgcacatctcgaccc

SEQ ID NO: 83 - DNA, 19 bp, Artificial Sequence
  Forward primer for Tmem26
accctgtcatcccacagag

SEQ ID NO: 84 - DNA, 22 bp, Artificial Sequence
  Reverse primer for Tmem26
tgtttggtggagtcctaaggtc

SEQ ID NO: 85 - DNA, 24 bp, Artificial Sequence
  Forward primer for Tph1
accatgattgaagacaacaaggag

SEQ ID NO: 86 - DNA, 20 bp, Artificial Sequence
  Reverse primer for Tph1
tcaactgttctcggctgatg

SEQ ID NO: 87 - DNA, 24 bp, Artificial Sequence
  Forward primer for Tph2
gccatgcagcccgcaatgatgatg

SEQ ID NO: 88 - DNA, 20 bp, Artificial Sequence
  Reverse primer for Tph2
caactgctgtcttgctgctc

SEQ ID NO: 89 - DNA, 21 bp, Artificial Sequence
  Forward primer for Ucp1
ctttgcctcactcaggattgg

SEQ ID NO: 90 - DNA, 21 bp, Artificial Sequence
  Reverse primer for Ucp1
ctttgcctcactcaggattgg

SEQ ID NO: 91 - DNA, 20 bp, Artificial Sequence
  Forward primer for Ucp2
gtggtcggagataccagagc

SEQ ID NO: 92 - DNA, 20 bp, Artificial Sequence
  Reverse primer for Ucp2
gaggttggctttcaggagag